SEQUENCE LISTING (16 sequences)

SEQ ID NO: 1 - RNA, 21 nt, Homo sapiens
uuccuuaacuaaaguacucag

SEQ ID NO: 2 - RNA, 22 nt, Homo sapiens
uuccuuaacuaaaguacucaga

SEQ ID NO: 3 - RNA, 22 nt, Homo sapiens
uuuccuuaacuaaaguacucag

SEQ ID NO: 4 - RNA, 23 nt, Homo sapiens
uuuccuuaacuaaaguacucaga

SEQ ID NO: 5 - RNA, 119 nt, Homo sapiens
auuucuuuccuuaacuaaaguacucagauauuuauccaaacauuauugcuaugggauuuccugcagaaagacuugaaggcguauacaggaacaauauugaugauguaguaagguaagaa

SEQ ID NO: 6 - skipped
  Synthetic

SEQ ID NO: 7 - DNA, 21 bp, Artificial sequence
  Synthetic
ctgagtactttagttaaggaa

SEQ ID NO: 8 - DNA, 22 bp, Artificial sequence
  Synthetic
tctgagtactttagttaaggaa

SEQ ID NO: 9 - DNA, 22 bp, Artificial sequence
  Synthetic
ctgagtactttagttaaggaaa

SEQ ID NO: 10 - DNA, 23 bp, Artificial sequence
  Synthetic
tctgagtactttagttaaggaaa

SEQ ID NO: 11 - DNA, 21 bp, Artificial sequence
  Synthetic
ttccttaactaaagtactcag

SEQ ID NO: 12 - DNA, 22 bp, Artificial sequence
  Synthetic
ttccttaactaaagtactcaga

SEQ ID NO: 13 - DNA, 22 bp, Artificial sequence
  Synthetic
tttccttaactaaagtactcag

SEQ ID NO: 14 - DNA, 23 bp, Artificial sequence
  Synthetic
tttccttaactaaagtactcaga

SEQ ID NO: 15 - DNA, 119 bp, Artificial sequence
  Synthetic
ttcttaccttactacatcatcaatattgttcctgtatacgccttcaagtctttctgcaggaaatcccatagcaataatgtttggataaatatctgagtactttagttaaggaaagaaat

SEQ ID NO: 16 - DNA, 119 bp, Artificial sequence
  Synthetic
atttctttccttaactaaagtactcagatatttatccaaacattattgctatgggatttcctgcagaaagacttgaaggcgtatacaggaacaatattgatgatgtagtaaggtaagaa

(19) United States
(12) Patent Application Publication
(10) Pub. No.: US 2016/0024586 A1
(43) Pub. Date: Jan. 28, 2016

(54) METHODS OF DETECTING CANCER

(71) Applicant: CEPHEID, Sunnyvale,, CA (US)

(72) Inventors: Olivier Delfour, Caraman (FR); David Vilanova, Caraman (FR)

(73) Assignee: Cepheid, Sunnyvale, CA (US)

(21) Appl. No.: 14/773,060

(22) PCT Filed: Mar. 10, 2014
(86) PCT No.: PCT/US2014/022544

§ 371 Date: Sep. 4, 2015

Related U.S. Application Data
(60) Provisional application No. 61/777,038, filed on Mar. 12, 2013.

(51) Int. Cl. C12Q 1/68

(52) U.S. Cl. CPC C12Q 1/6886; C12Q 2600/118; C12Q 2600/112

(57) ABSTRACT

Methods of detecting cancer are provided. Methods of detecting changes in the levels of one or more small RNAs associated with cancer are also provided. Compositions and kits are also provided.

### 1. BACKGROUND

The importance of the physiological function of phosphatase and tensin homologue (PTEN) is illustrated by its frequent disruption in cancer. By suppressing the phosphoinositide 3-kinase (PI3K)-AKT-mammalian target of rapamycin (mTOR) pathway through its lipid phosphatase activity, PTEN governs many cellular processes including survival, proliferation, energy metabolism and cellular architecture. See, e.g., Hollander et al., 2011, Nat. Rev. Cancer, 11: 289-301. Many mechanisms regulating PTEN expression and function, including transcriptional regulation, post-transcriptional regulation by non-coding RNAs, post-translational modifications and protein-protein interactions, are altered in cancer.

MicroRNAs are post transcriptional regulators of gene expression, and may provide an important layer of genetic regulation in tumorigenesis, making them viable therapeutic targets and diagnostic markers.

There remains a need for molecular markers in cancer.

### 2. SUMMARY

In some embodiments, methods for detecting the presence of cancer in a subject are provided. In some embodiments, methods for monitoring therapy in a cancer patient are provided. In some embodiments, a method comprises detecting the level of 13214 in a sample from the subject. In some embodiments, a method comprises comparing the level of the 13214 in the sample to a normal level of 13214. In some embodiments, detection of a level of 13214 that is lower than a normal level of 13214, indicates the presence of cancer in the subject.

In some embodiments, methods of facilitating the diagnosis of cancer in a subject are provided. Methods of monitoring therapy in a cancer patient are also provided. In some embodiments, a method comprises detecting the level of 13214 in a sample from the subject. In some embodiments, a method comprises communicating the results of the detection to a medical practitioner for the purpose of determining whether the subject has cancer. In some embodiments, a method comprises communicating the results of the detection to a medical practitioner for the purpose of monitoring therapy in the cancer patient.

In some embodiments, methods of monitoring response to therapy in a cancer patient are provided. In some embodiments, a method comprises detecting the level of 13214, in a first sample from the subject taken at a first time point. In some embodiments, a method comprises comparing the level of 13214 to the level of 13214 in a second sample from the patient taken at a second time point, wherein the second time point is prior to the first time point. In some embodiments, an increase in the level of 13214 in the first sample relative to the second sample, indicates that the cancer patient is responding to therapy.

In some embodiments, methods for detecting the presence of cancer in a subject are provided, comprising obtaining a sample from the subject and providing the sample to a laboratory for detection of the level of 13214 in the sample. In some embodiments, a method comprises receiving from the laboratory a communication indicating the level of 13214. In some embodiments, detection of a level of 13214 that is lower than a normal level of 13214, indicates the presence of cancer in the subject.

In some embodiments, methods for monitoring response to therapy in a cancer patient are provided, comprising obtaining a first sample from the subject at a first time point and providing the first sample to a laboratory for detection of the level of 13214 in the sample. In some embodiments, a method comprises receiving from the laboratory a communication indicating the level of 13214. In some embodiments, a method comprises comparing the level of 13214 in the first sample to the level of 13214 in a second sample that was taken at a second time point, wherein the second time point is prior to the first time point. In some embodiments, an increase in the level of 13214 in the first sample relative to the second sample, indicates that the cancer patient is responding to therapy.

In any of the embodiments described herein, the detecting may comprise hybridizing at least one polynucleotide comprising at least 8 contiguous nucleotides, at least 10 contiguous nucleotides, at least 12 contiguous nucleotides, at least 14 contiguous nucleotides, or at least 16 contiguous nucleotides of a sequence selected from SEQ ID NOs: 7 to 14 to RNA from the sample or cDNA reverse-transcribed from RNA from the sample. In some embodiments, a method comprises detecting a complex comprising a polynucleotide and a 13214 RNA or cDNA reverse transcribed therefrom.

In any of the embodiments described herein, 13214 may be selected from mature 13214, a mature 13214 isomir, pre-13214, and combinations thereof. In any of the embodiments described herein, 13214 may be 13214-L. In any of the embodiments described herein, 13214 may have a sequence selected from SEQ ID NOs: 1 to 4.

In any of the embodiments described herein, the sample may be selected from a tissue sample and a bodily fluid. In any of the embodiments described herein, the bodily fluid may be selected from blood, urine, sputum, saliva, mucus, and semen. In any of the embodiments described herein, the sample may be a blood sample. In any of the embodiments described herein, the sample may be a serum sample. In any of the embodiments described herein, the sample may be a plasma sample.

In any of the embodiments described herein, the cancer may be selected from breast cancer, endometrial cancer, uterine cancer, ovarian cancer, cervical cancer, prostate cancer, leukemia, lymphoma, glioma, glioblastoma, melanoma, lung cancer, non-small cell lung cancer, liver cancer, bladder cancer, kidney cancer, pancreatic cancer, stomach cancer, adrenal pheochromocytoma, colon cancer, intestinal cancer, thyroid cancer, and skin cancer. In any of the embodiments described herein, the cancer may be a leukemia. In some embodiments, the leukemia is selected from acute lymphoblastic leukemia and acute myeloblastic leukemia.

In any of the embodiments described herein, the detecting may comprise quantitative RT-PCR.

In some embodiments, use of 13214 is provided for detecting the presence of cancer in a subject, or for monitoring therapy in a cancer patient.

In some embodiments, an oligonucleotide is provided that comprises at least eight contiguous nucleotides that are complementary to 13214, wherein the oligonucleotide is between 8 and 200, between 8 and 150, between 8 and 100, between 8 and 75, between 8 and 50, between 8 and 40, or between 8 and 30 nucleotides long, for detecting cancer in a subject. In some embodiments, an oligonucleotide is provided that comprises at least eight contiguous nucleotides that are complementary to a cDNA reverse-transcribed from 13214, wherein the oligonucleotide is between 8 and 200, between 8 and 150, between 8 and 100, between 8 and 75, between 8 and 50, between 8 and 40, or between 8 and 30 nucleotides long, for detecting cancer in a subject.

Further embodiments and details of the inventions are described below.

### 3. BRIEF DESCRIPTION OF THE FIGURES

FIG. 1A-B shows (A) a plot of qRT-PCR Ct values for 13214 in serum samples from various cancer patients and healthy individuals and (B) a receiver operating characteristic plot of the data in (A), as described in Example 1.

FIG. 2 shows a plot of qRT-PCR Ct values for 13214 in serum samples from patients with acute lymphoblastic leukemia (ALL) acute myeloblastic leukemia (AML), other cancers, solid tumors, and healthy individuals, as described in Example 1.

### 4. DETAILED DESCRIPTION

### 4.1. Detecting Cancer

4.1.1. General Methods

Alterations in the PTEN gene and/or alterations in PTEN expression have been found in many cancers. See, e.g., Hollander et al., 2011, Nat. Rev. Cancer, 11: 289-301. The present inventors have identified a microRNA, 13214, which is located in the PTEN gene, overlapping with the beginning of exon 2. The present inventors have demonstrated that 13214 levels are reduced in various cancers, including cancers that have been shown to involve PTEN deletions and/or mutations, relative to the levels in healthy individuals.

Methods for detecting human cancer are provided. In some embodiments, methods for detecting cancer are provided. In some embodiments, the cancer is selected from breast cancer, endometrial cancer, uterine cancer, ovarian cancer, cervical cancer, prostate cancer, leukemia, lymphoma, glioma, glioblastoma, melanoma, lung cancer, non-small cell lung cancer, liver cancer, bladder cancer, kidney cancer, pancreatic cancer, stomach cancer, adrenal pheochromocytoma, colon cancer, intestinal cancer, thyroid cancer, and skin cancer. In some embodiments, methods of detecting leukemia are provided. In some embodiments, the leukemia is selected from acute myeloblastic leukemia and acute lymphoblastic leukemia. In some embodiments, methods for detecting early stage cancer that is likely to progress are provided.

In some embodiments, a method of detecting cancer comprises detecting 13214 in a sample from a patient. In some embodiments, the method comprises detecting a below-normal level of 13214 in a sample from a patient. In some embodiments, in any of the methods described herein, 13214 is mature 13214.

In some embodiments, the level of one or more RNAs is determined in serum. In some embodiments, the method further comprises detecting an above-normal level of at least one additional target RNA. In some embodiments, the method further comprises detecting a below-normal level of at least one additional target RNA. In some embodiments, the method comprises detecting mature microRNA and pre-microRNA. In some embodiments, the method comprises detecting mature microRNA.

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine. Thus, where a microRNA sequence includes “T”, one skilled in the art would understand that that position in the native microRNA is a likely uracil.

As used herein, the term “13214” includes pre-13214, mature 13214 (13214-L), mature 13214 isomirs, 13214* (13214-R), and any other RNAs formed through processing of the pre-13214, as well as any of products of pre-13214 after eventual post-transcriptional modification or editing. Mature 13214 (also referred to as “13214-L”) has the sequence:

(SEQ ID NO: 1)
5′-UUCCUUAACUAAAGUACUCAG-3′.

Pre-13214, which is the pre-microRNA form of 13214, has the sequence:

(SEQ ID NO: 5)
5′-AUUUCUUUCC UUAACUAAAG UACUCAGAUA UUUAUCCAAA
CAUUAUUGCU AUGGGAUUUC CUGCAGAAAG ACUUGAAGGC
GUAUACAGGA ACAAUAUUGA UGAUGUAGUA AGGUAAGAA-3′.

Other exemplary 13214 sequences include:

(SEQ ID NO: 2)
5′-UUCCUUAACUAAAGUACUCAGA-3′;
(SEQ ID NO: 3)
5′-UUUCCUUAACUAAAGUACUCAG-3′;
(SEQ ID NO: 4)
5′-UUUCCUUAACUAAAGUACUCAGA-3′;

As demonstrated in the Examples, at least mature 13214 was detected at reduced levels in certain cancer patients, using, e.g., quantitative RT-PCT.

In the present disclosure, the term “target RNA” is used for convenience to refer to 13214 and also to other target RNAs. Thus, it is to be understood that when a discussion is presented in terms of a target RNA, that discussion is specifically intended to encompass 13214 and/or other target RNAs.

In some embodiments, detection of a level of target RNA that is greater than a normal level of target RNA indicates the presence of cancer in the sample. In some embodiments, detection of a level of target RNA that is less than a normal level of target RNA indicates the presence of cancer in the sample. In some embodiments, detection of a level of 13214 that is less than a normal level of 13214 indicates the presence of cancer in the sample. In some embodiments, the detecting is done quantitatively. In other embodiments, the detecting is done qualitatively. In some embodiments, detecting a target RNA comprises forming a complex comprising a polynucleotide and a nucleic acid selected from a target RNA, a DNA amplicon of a target RNA, and a complement of a target RNA. In some embodiments, the level of the complex is then detected and compared to a normal level of the same complex.

Exemplary cancers that may be detected by measuring levels of 13214 include, but are not limited to, breast cancer, endometrial cancer, uterine cancer, ovarian cancer, cervical cancer, prostate cancer, leukemia, lymphoma, glioma, glioblastoma, melanoma, lung cancer (such as non-small cell lung cancer), liver cancer, bladder cancer, kidney cancer, pancreatic cancer, stomach cancer, adrenal pheochromocytoma, colon cancer, intestinal cancer, thyroid cancer, and skin cancer.

Cancer can be divided into clinical and pathological stages. The clinical stage is based on all available information about a tumor, such as information gathered through physical examination, radiological examination, endoscopy, etc. The pathological stage is based on the microscopic pathology of a tumor.

The TNM (tumor, node, metastasis) system classifies a cancer by three parameters—the size of the tumor and whether it has invaded nearby tissues, involvement of lymph nodes, and metastases. T (tumor) is assigned a number from 1 to 4, according to the size and extent of the primary tumor. N (node) is assigned a number from 0 to 3, in which 0 means no spreading to the lymph nodes, 1 is spreading to the closest lymph nodes, and 3 is spreading to the most distant and greatest number of lymph nodes, and 2 is intermediate between 1 and 3. M (metastasis) is assigned 0 for no distant metastases, or 1 for distant metastases beyond regional lymph nodes.

Mature human microRNAs are typically composed of 17-27 contiguous ribonucleotides, and often are 21 or 22 nucleotides in length. While not intending to be bound by theory, mammalian microRNAs mature as described herein. A gene coding for a microRNA is transcribed, leading to production of a microRNA precursor known as the “pri-microRNA” or “pri-miRNA.” The pri-miRNA can be part of a polycistronic RNA comprising multiple pri-miRNAs. In some circumstances, the pri-miRNA forms a hairpin with a stem and loop, which may comprise mismatched bases. The hairpin structure of the pri-miRNA is recognized by Drosha, which is an RNase III endonuclease protein. Drosha can recognize terminal loops in the pri-miRNA and cleave approximately two helical turns into the stem to produce a 60-70 nucleotide precursor known as the “pre-microRNA” or “pre-miRNA.” Drosha can cleave the pri-miRNA with a staggered cut typical of RNase III endonucleases yielding a pre-miRNA stem loop with a 5′ phosphate and an approximately 2-nucleotide 3′ overhang. Approximately one helical turn of the stem (about 10 nucleotides) extending beyond the Drosha cleavage site can be essential for efficient processing. The pre-miRNA is subsequently actively transported from the nucleus to the cytoplasm by Ran-GTP and the export receptor Exportin-5.

The pre-miRNA can be recognized by Dicer, another RNase III endonuclease. In some circumstances, Dicer recognizes the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and an approximately 2-nucleotide 3′ overhang. The resulting siRNA-like duplex, which may comprise mismatches, comprises the mature microRNA and a similar-sized fragment known as the microRNA*. The microRNA and microRNA* may be derived from opposing arms of the pri-miRNA and pre-miRNA. The mature microRNA is then loaded into the RNA-induced silencing complex (“RISC”), a ribonucleoprotein complex. In some cases, the microRNA* also has gene silencing or other activity.

Nonlimiting exemplary small cellular RNAs include, in addition to microRNAs, small nuclear RNAs, tRNAs, ribosomal RNAs, snoRNAs, piRNAs, siRNAs, and small RNAs formed by processing any of those RNAs. In some embodiments, a target RNA is a small cellular RNA.

In some embodiments, a target RNA, such as 13214, can be measured in samples collected at one or more times from a patient to monitor the status or progress of cancer in the patient.

In some embodiments, the sample to be tested is a bodily fluid, such as blood, sputum, mucus, saliva, urine, semen, etc. In some embodiments, a sample to be tested is a blood sample. In some embodiments, the blood sample is whole blood. In some embodiments, the blood sample is a sample of blood cells. In some embodiments, the blood sample is plasma. In some embodiments, the blood sample is serum. In some embodiments, the methods described herein are used for early detection of cancer in a sample of blood or serum.

The clinical sample to be tested is, in some embodiments, freshly obtained. In other embodiments, the sample is a fresh frozen specimen. In some embodiments, the sample is a tissue sample, such as a formalin-fixed paraffin embedded sample. In some embodiments, the sample is a liquid cytology sample.

Thus, in some embodiments, methods described herein can be used for routine screening of healthy individuals with no risk factors. In some embodiments, methods described herein are used to screen asymptomatic individuals having one or more risk factors.

In some embodiments, the methods described herein can be used to assess the effectiveness of a treatment for cancer in a patient. In some embodiments, target RNA levels, such as 13214, are determined at various times during the treatment, and are compared to target RNA levels from an archival sample taken from the patient before the manifestation of any signs of cancer or before beginning treatment. In some embodiments, target RNA levels are compared to target RNA levels from an archival sample of normal tissue taken from the patient or a sample of tissue taken from a tumor-free part of the patient's body. Ideally, target RNA levels in the normal sample evidence no aberrant changes in target RNA levels. Thus, in such embodiments, the progress of treatment of an individual with cancer can be assessed by comparison to a sample from the same individual when he was healthy or prior to beginning treatment, or by comparison to a sample of healthy cells from the same individual.

In some embodiments, use of 13214 for monitoring the response of a cancer patient to therapy is provided. In the monitoring to therapy, preferably a blood sample, such as serum, is used. In the monitoring of therapy, the level of 13214 is assessed against its baseline level determined at the initiation of therapy. In some embodiments, changes from the baseline level indicates response to therapy where the level of 13214 increases. In some embodiments, a change from the baseline level indicates resistance to therapy where the level of 13214 decreases.

In some embodiments, a method comprises detecting 13214. In some embodiments, in combination with detecting 13214, a method further comprises detecting at least one additional target RNA. Such additional target RNAs include, but are not limited to, other microRNAs, small cellular RNAs, and mRNAs.

In embodiments in which the method comprises detecting levels of at least two RNAs, including 13214, the levels of a plurality of RNAs may be detected concurrently or simultaneously in the same assay reaction. In some embodiments, RNA levels are detected concurrently or simultaneously in separate assay reactions. In some embodiments, RNA levels are detected at different times, e.g., in serial assay reactions.

In some embodiments, a method comprises detecting the level of 13214 in a sample from a subject, wherein detection of a level of 13214 that is less than a normal level of the RNA indicates the presence of cancer in the subject.

In some embodiments, a method of facilitating diagnosis of cancer in a subject is provided. Such methods comprise detecting the level of 13214 in a sample from the subject. In some embodiments, information concerning the level of 13214 in the sample from the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, detecting the level of 13214 is carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc.

In some embodiments, methods of detecting the presence cancer are provided. In some embodiments, methods of diagnosing cancer are provided. In some embodiments, the method comprises obtaining a sample from a subject and providing the sample to a laboratory for detection of the level of 13214 in the sample. In some embodiments, the method further comprises receiving a communication from the laboratory that indicates the level of 13214 in the sample. In some embodiments, cancer is present if the level of 13214 in the sample is less than a normal level of 13214. A “laboratory,” as used herein, is any facility that detects the level of 13214 in a sample by any method, including the methods described herein, and communicates the level to a medical practitioner. In some embodiments, a laboratory is under the control of a medical practitioner. In some embodiments, a laboratory is not under the control of the medical practitioner.

When a laboratory communicates the level of 13214 to a medical practitioner, in some embodiments, the laboratory communicates a numerical value representing the level of 13214 in the sample, with or without providing a numerical value for a normal level. In some embodiments, the laboratory communicates the level of 13214 by providing a qualitative value, such as “high,” “low,” “elevated,” “decreased,” etc.

As used herein, when a method relates to detecting cancer, determining the presence of cancer, and/or diagnosing cancer, the method includes activities in which the steps of the method are carried out, but the result is negative for the presence of cancer. That is, detecting, determining, and diagnosing cancer include instances of carrying out the methods that result in either positive or negative results (e.g., whether 13214 levels are normal or less than normal).

As used herein, the term “subject” means a human. In some embodiments, the methods described herein may be used on samples from non-human animals.

The common, or coordinate, expression of target RNAs that are physically proximal to one another in the genome permits the informative use of such chromosome-proximal target RNAs in methods herein.

The coding sequence for 13214 is located on chromosome 10 at 10q23.31, overlapping with exon 2 of the PTEN gene. In some embodiments, the level of expression of one or more target RNAs located within about 1 kilobase (kb), within about 2 kb, within about 5 kb, within about 10 kb, within about 20 kb, within about 30 kb, within about 40 kb, and even within about 50 kb of the chromosomal location of 13214 is detected in lieu of, or in addition to, measurement of expression of 13214 in the methods described herein. See Baskerville, S. and Bartel D. P. (2005) RNA 11:241-247.

In some embodiments, the methods further comprise detecting in a sample the expression of at least one target RNA gene located in close proximity to chromosomal features, such as cancer-associated genomic regions, fragile sites, and human papilloma virus integration sites.

In some embodiments, more than RNA is detected simultaneously in a single reaction. In some embodiments, at least 2, at least 3, at least 5, or at least 10 RNAs are detected simultaneously in a single reaction. In some embodiments, all RNAs are detected simultaneously in a single reaction.

4.1.2. Exemplary Controls

In some embodiments, a normal level (a “control”) of a target RNA, such as 13214, can be determined as an average level or range that is characteristic of normal cells or other reference material, against which the level measured in the sample can be compared. The determined average or range of a target RNA in normal subjects can be used as a benchmark for detecting above-normal levels of the target RNA that are indicative of cancer. In some embodiments, normal levels of a target RNA can be determined using individual or pooled RNA-containing samples from one or more individuals.

In some embodiments, determining a normal level of a target RNA, such as 13214, comprises detecting a complex comprising a polynucleotide for detection hybridized to a nucleic acid selected from a target RNA, a DNA amplicon of the target RNA, and a complement of the target RNA. That is, in some embodiments, a normal level can be determined by detecting a DNA amplicon of the target RNA, or a complement of the target RNA rather than the target RNA itself. In some embodiments, a normal level of such a complex is determined and used as a control. The normal level of the complex, in some embodiments, correlates to the normal level of the target RNA. Thus, when a normal level of a target is discussed herein, that level can, in some embodiments, be determined by detecting such a complex.

In some embodiments, a control comprises RNA from cells of a single individual, e.g., from normal tissue of a patient undergoing surgical resection for cancer. In some embodiments, a control comprises RNA from blood, such as whole blood or serum, of a single individual. In some embodiments, a control comprises RNA from a pool of cells from multiple individuals. In some embodiments, a control comprises RNA from a pool of blood, such as whole blood or serum, from multiple individuals. In some embodiments, a control comprises commercially-available human RNA, such as, for example, human tissue total RNA (many available from Ambion). In some embodiments, a normal level or normal range has already been predetermined prior to testing a sample for an elevated or reduced level.

In some embodiments, the normal level of a target RNA, such as 13214, can be determined from one or more continuous cell lines, typically cell lines previously shown to have levels of RNAs that approximate the levels in normal cells.

In some embodiments, a method comprises detecting the level of 13214. In some embodiment, in addition to detecting the level of 13214, a method comprises detecting the level of at least one additional target RNA. In some embodiments, a method further comprises comparing the level of 13214 to a normal level of the at least one RNA. In some embodiments, a method further comprises comparing the level of at least one target RNA to a control level of the at least one target RNA. A control level of a target RNA is, in some embodiments, the level of the target RNA in a normal cell. A control level of a target RNA is, in some embodiments, the level of the target RNA in a serum from a healthy individual. In some such embodiments, a control level may be referred to as a normal level.

In some embodiments, a reduced level of 13214 in a sample relative to the level of 13214 in normal cells or normal serum indicates cancer.

In some embodiments, a greater level of at least one additional target RNA relative to the level of the at least one additional target RNA in a normal cell indicates cancer. In some embodiments, a lower level of at least one additional target RNA relative to the level of the at least one additional target RNA in a normal cell indicates cancer.

In some embodiments, the level of a target RNA, such as 13214, is compared to a reference level, e.g., from a confirmed cancer. In some such embodiments, a similar level of a target RNA relative to the reference sample indicates cancer.

In some embodiments, a level of 13214 that is at least about two-fold less than a normal level of 13214 indicates the presence of cancer. In various embodiments, a level of 13214 that is at least about 3-fold, at least about 4-fold, at least about 5-fold, at least about 6-fold, at least about 7-fold, at least about 8-fold, at least about 9-fold, or at least about 10-fold less than the level of 13214 in a control sample indicates the presence of cancer. In various embodiments, a level of 13214 that is at least about 3-fold, at least about 4-fold, at least about 5-fold, at least about 6-fold, at least about 7-fold, at least about 8-fold, at least about 9-fold, or at least about 10-fold less than a normal level of 13214 indicates the presence of cancer.

In some embodiments, a control level of a target RNA, such as 13214, is determined contemporaneously, such as in the same assay or batch of assays, as the level of the target RNA in a sample. In some embodiments, a control level of a target RNA, such as 13214, is not determined contemporaneously as the level of the target RNA in a sample. In some such embodiments, the control level has been determined previously.

In some embodiments, the level of a target RNA is not compared to a control level, for example, when it is known that the target RNA is present at very low levels, or not at all, in normal cells. In such embodiments, detection of a high level of the target RNA in a sample is indicative of cancer. Similarly, in some embodiments, if a target RNA is present at high levels in normal cells or normal serum, the detection of a very low level in a sample is indicative of cancer.

4.1.3. Exemplary Methods of Preparing RNAs

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen™), Total RNA Extraction Kit (iNtRON Biotechnology), Total RNA Purification Kit (Norgen Biotek Corp.), RNAqueous™ (Ambion), MagMAX™ (Ambion), RecoverAll™ (Ambion), RNeasy (Qiagen), etc.

In some embodiments, small RNAs are isolated or enriched. In some embodiments “small RNA” refers to RNA molecules smaller than about 200 nucleotides (nt) in length. In some embodiments, “small RNA” refers to RNA molecules smaller than about 100 nt, smaller than about 90 nt, smaller than about 80 nt, smaller than about 70 nt, smaller than about 60 nt, smaller than about 50 nt, or smaller than about 40 nt.

Enrichment of small RNAs can be accomplished by method. Such methods include, but are not limited to, methods involving organic extraction followed by adsorption of nucleic acid molecules on a glass fiber filter using specialized binding and wash solutions, and methods using spin column purification. Enrichment of small RNAs may be accomplished using commercially-available kits, such as mirVana™ Isolation Kit (Ambion), mirPremier™ microRNA Isolation Kit (Sigma-Aldrich), PureLink™ miRNA Isolation Kit (Invitrogen), miRCURY™ RNA isolation kit (Exiqon), microRNA Purification Kit (Norgen Biotek Corp.), miRNeasy kit (Qiagen), etc. In some embodiments, purification can be accomplished by the TRIzol® (Invitrogen) method, which employs a phenol/isothiocyanate solution to which chloroform is added to separate the RNA-containing aqueous phase. Small RNAs are subsequently recovered from the aqueous by precipitation with isopropyl alcohol. In some embodiments, small RNAs can be purified using chromatographic methods, such as gel electrophoresis using the flashPAGE™ Fractionator available from Applied Biosystems.

In some embodiments, small RNA is isolated from other RNA molecules to enrich for target RNAs, such that the small RNA fraction (e.g., containing RNA molecules that are 200 nucleotides or less in length, such as less than 100 nucleotides in length, such as less than 50 nucleotides in length, such as from about 10 to about 40 nucleotides in length) is substantially pure, meaning it is at least about 80%, 85%, 90%, 95% pure or more, but less than 100% pure, with respect to larger RNA molecules. Alternatively, enrichment of small RNA can be expressed in terms of fold-enrichment. In some embodiments, small RNA is enriched by about, at least about, or at most about 5×, 10×, 20×, 30×, 40×, 50×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein, with respect to the concentration of larger RNAs in an RNA isolate or total RNA in a sample.

In some embodiments, RNA levels are measured in a sample in which RNA has not first been purified from the cells. In some embodiments, RNA levels are measured in a sample in which RNA has been isolated, but not enriched for small RNAs.

In some embodiments, RNA is modified before a target RNA, such as 13214, is detected. In some embodiments, the modified RNA is total RNA. In other embodiments, the modified RNA is small RNA that has been purified from total RNA or from cell lysates, such as RNA less than 200 nucleotides in length, such as less than 100 nucleotides in length, such as less than 50 nucleotides in length, such as from about 10 to about 40 nucleotides in length. RNA modifications that can be utilized in the methods described herein include, but are not limited to, the addition of a poly-dA or a poly-dT tail, which can be accomplished chemically or enzymatically, and/or the addition of a small molecule, such as biotin.

In some embodiments, a target RNA, such as 13214, is reverse transcribed. In some embodiments, cDNA is modified when it is reverse transcribed, such as by adding a poly-dA or a poly-dT tail during reverse transcription. In other embodiments, RNA is modified before it is reverse transcribed. In some embodiments, total RNA is reverse transcribed. In other embodiments, small RNAs are isolated or enriched before the RNA is reverse transcribed.

When a target RNA, such as 13214, is reverse transcribed, a complement of the target RNA is formed. In some embodiments, the complement of a target RNA is detected rather than a target RNA itself (or a DNA copy thereof). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In such embodiments, a polynucleotide for detection comprises at least a portion that is identical in sequence to the target RNA, although it may contain thymidine in place of uridine, and/or comprise other modified nucleotides.

In some embodiments, the method of detecting a target RNA, such as 13214, comprises amplifying cDNA complementary to the target RNA. Such amplification can be accomplished by any method. Exemplary methods include, but are not limited to, real time PCR, endpoint PCR, and amplification using T7 polymerase from a T7 promoter annealed to a cDNA, such as provided by the SenseAmp Plus™ Kit available at Implen, Germany.

When a target RNA or a cDNA complementary to a target RNA is amplified, in some embodiments, a DNA amplicon of the target RNA is formed. A DNA amplicon may be single stranded or double-stranded. In some embodiments, when a DNA amplicon is single-stranded, the sequence of the DNA amplicon is related to the target RNA in either the sense or antisense orientation. In some embodiments, a DNA amplicon of a target RNA is detected rather than the target RNA itself. Thus, when the methods discussed herein indicate that a target RNA is detected, or the level of a target RNA is determined, such detection or determination may be carried out on a DNA amplicon of the target RNA instead of, or in addition to, the target RNA itself. In some embodiments, when the DNA amplicon of the target RNA is detected rather than the target RNA, a polynucleotide for detection is used that is complementary to the complement of the target RNA. In some embodiments, when the DNA amplicon of the target RNA is detected rather than the target RNA, a polynucleotide for detection is used that is complementary to the target RNA. Further, in some embodiments, multiple polynucleotides for detection may be used, and some polynucleotides may be complementary to the target RNA and some polynucleotides may be complementary to the complement of the target RNA.

In some embodiments, the method of detecting one or more target RNAs, including 13214, as described below. In some embodiments, detecting one or more target RNAs comprises real-time monitoring of an RT-PCR reaction, which can be accomplished by any method. Such methods include, but are not limited to, the use of TaqMan®, Molecular beacon, or Scorpion probes (i.e., FRET probes) and the use of intercalating dyes, such as SYBR green, EvaGreen, thiazole orange, YO-PRO, TO-PRO, etc.

4.1.4. Exemplary Analytical Methods

As described above, methods are presented for detecting cancer. In some embodiments, the method comprises detecting a level of 13214. In some embodiments, the method further comprises detecting a level of at least one additional target RNA.

In some embodiments, a method comprises detecting a level of a target RNA, such as 13214, that is lower in the sample than a normal level of the target RNA in a control sample, such as a sample derived from normal cells or normal serum. In some embodiments, 13214 is mature 13214. In some embodiments, a target RNA, in its mature form, comprises fewer than 30 nucleotides. In some embodiments, a target RNA is a microRNA. In some embodiments, a target RNA is a small cellular RNA.

In some embodiments, in addition to detecting a level of 13214, a method further comprises detecting a level of at least one target RNA of the human miRNome. As used herein, the term “human miRNome” refers to all microRNA genes in a human cell and the mature microRNAs produced therefrom.

Any analytical procedure capable of permitting specific and quantifiable (or semi-quantifiable) detection of a target RNA, such as 13214, may be used in the methods herein presented. Such analytical procedures include, but are not limited to, the microarray methods and the RT-PCR methods set forth in the Examples, and methods known to those skilled in the art.

In some embodiments, detection of a target RNA, such as 13214, comprises forming a complex comprising a polynucleotide that is complementary to a target RNA or to a complement thereof, and a nucleic acid selected from the target RNA, a DNA amplicon of the target RNA, and a complement of the target RNA. Thus, in some embodiments, the polynucleotide forms a complex with a target RNA. In some embodiments, the polynucleotide forms a complex with a complement of the target RNA, such as a cDNA that has been reverse transcribed from the target RNA. In some embodiments, the polynucleotide forms a complex with a DNA amplicon of the target RNA. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target RNA, complement of the target RNA, or DNA amplicon of the target RNA. The polynucleotide, in some embodiments, is a primer or probe.

In some embodiments, a method comprises detecting the complex. In some embodiments, the complex does not have to be associated at the time of detection. That is, in some embodiments, a complex is formed, the complex is then dissociated or destroyed in some manner, and components from the complex are detected. An example of such a system is a TaqMan® assay. In some embodiments, when the polynucleotide is a primer, detection of the complex may comprise amplification of the target RNA, a complement of the target RNA, or a DNA amplicon of a target RNA.

In some embodiments the analytical method used for detecting at least one target RNA, including 13214, in the methods set forth herein includes real-time quantitative RT-PCR. See Chen, C. et al. (2005) Nucl. Acids Res. 33:e179 and PCT Publication No. WO 2007/117256, which are incorporated herein by reference in its entirety. In some embodiments, the analytical method used for detecting at least one target RNA includes the method described in U.S. Publication No. US2009/0123912 A1, which is incorporated herein by reference in its entirety. In an exemplary method described in that publication, an extension primer comprising a first portion and second portion, wherein the first portion selectively hybridizes to the 3′ end of a particular small RNA and the second portion comprises a sequence for universal primer, is used to reverse transcribe the small RNA to make a cDNA. A reverse primer that selectively hybridizes to the 5′ end of the small RNA and a universal primer are then used to amplify the cDNA in a quantitative PCR reaction.

In some embodiments, the analytical method used for detecting at least one target RNA, including 13214, includes the use of a TaqMan® probe. In some embodiments, the analytical method used for detecting at least one target RNA includes a TaqMan® assay, such as the TaqMan® MicroRNA Assays sold by Applied Biosystems, Inc. In an exemplary TaqMan® assay, total RNA is isolated from the sample. In some embodiments, the assay can be used to analyze about 10 ng of total RNA input sample, such as about 9 ng of input sample, such as about 8 ng of input sample, such as about 7 ng of input sample, such as about 6 ng of input sample, such as about 5 ng of input sample, such as about 4 ng of input sample, such as about 3 ng of input sample, such as about 2 ng of input sample, and even as little as about 1 ng of input sample containing small RNAs.

The TaqMan® assay utilizes a stem-loop primer that is specifically complementary to the 3′-end of a target RNA. In an exemplary TaqMan® assay, hybridizing the stem-loop primer to the target RNA is followed by reverse transcription of the target RNA template, resulting in extension of the 3′ end of the primer. The result of the reverse transcription is a chimeric (DNA) amplicon with the step-loop primer sequence at the 5′ end of the amplicon and the cDNA of the target RNA at the 3′ end. Quantitation of the target RNA is achieved by real time RT-PCR using a universal reverse primer having a sequence that is complementary to a sequence at the 5′ end of all stem-loop target RNA primers, a target RNA-specific forward primer, and a target RNA sequence-specific TaqMan® probe.

The assay uses fluorescence resonance energy transfer (“FRET”) to detect and quantitate the synthesized PCR product. Typically, the TaqMan® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TaqMan® probe is bound, the 5′-nuclease of the polymerase cleaves the probe, decoupling the dye and the quencher so that FRET is abolished and a fluorescence signal is generated. Fluorescence increases with each RT-PCR cycle proportionally to the amount of probe that is cleaved.

Additional exemplary methods for RNA detection and/or quantification are described, e.g., in U.S. Publication No. US 2007/0077570 (Lao et al.), PCT Publication No. WO 2007/025281 (Tan et al.), U.S. Publication No. US2007/0054287 (Bloch), PCT Publication No. WO2006/0130761 (Bloch), and PCT Publication No. WO 2007/011903 (Lao et al.), which are incorporated by reference herein in their entireties for any purpose.

In some embodiments, quantitation of the results of real-time RT-PCR assays is done by constructing a standard curve from a nucleic acid of known concentration and then extrapolating quantitative information for target RNAs of unknown concentration. In some embodiments, the nucleic acid used for generating a standard curve is an RNA (e.g., a microRNA or other small RNA) of known concentration. In some embodiments, the nucleic acid used for generating a standard curve is a purified double-stranded plasmid DNA or a single-stranded DNA generated in vitro.

In some embodiments, where the amplification efficiencies of the target nucleic acids and the endogenous reference are approximately equal, quantitation is accomplished by the comparative Ct (cycle threshold, e.g., the number of PCR cycles required for the fluorescence signal to rise above background) method. Ct values are inversely proportional to the amount of nucleic acid target in a sample. In some embodiments, Ct values of a target RNA, such as 13214, can be compared with a control or calibrator, such as RNA (e.g., a microRNAs or other small RNA) from normal tissue. In some embodiments, the Ct values of the calibrator and the target RNA are normalized to an appropriate endogenous housekeeping gene. In some embodiments, a threshold Ct (or a “cutoff Ct”) value for a target RNA, such as 13214, above which cancer is indicated, has previously been determined. In such embodiments, a control sample may not be assayed concurrently with the test sample.

In addition to the TaqMan® assays, other real-time RT-PCR chemistries useful for detecting and quantitating PCR products in the methods presented herein include, but are not limited to, Molecular Beacons, Scorpion probes and intercalating dyes, such as SYBR Green, EvaGreen, thiazole orange, YO-PRO, TO-PRO, etc., which are discussed below.

In some embodiments, real-time RT-PCR detection is performed specifically to detect and quantify the level of a single target RNA. The target RNA, in some embodiments, is 13214.

As described herein, in some embodiments, in addition to detecting the level of 13214, the level of at least one additional target RNA is detected.

In various other embodiments, real-time RT-PCR detection is utilized to detect, in a single multiplex reaction, at least 2, at least 3, at least 4, at least 5, at least 6, at least 7, or at least 8 target RNAs, including 13214.

In some multiplex embodiments, a plurality of probes, such as TaqMan® probes, each specific for a different RNA target, is used. In some embodiments, each target RNA-specific probe is spectrally distinguishable from the other probes used in the same multiplex reaction.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time RT-PCR is performed using any RT-PCR instrumentation available in the art. Typically, instrumentation used in real-time RT-PCR data collection and analysis comprises a thermal cycler, optics for fluorescence excitation and emission collection, and optionally a computer and data acquisition and analysis software.

In some embodiments, the analytical method used in the methods described herein is a DASL® (cDNA-mediated Annealing, Selection, Extension, and Ligation) Assay, such as the MicroRNA Expression Profiling Assay available from Illumina, Inc. (See www.illumina.com/downloads/MicroRNAAssayWorkflow.pdf). In some embodiments, total RNA is isolated from a sample to be analyzed by any method. Additionally, in some embodiments, small RNAs are isolated from a sample to be analyzed by any method. Total RNA or isolated small RNAs may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is complementary to at least 8, at least 9, at least 10, at least 11, at least 12, at least 13, at least 14, at least 15, at least 16, at least 17, at least 18, at least 19 contiguous nucleotides of 13214. In some DASL® embodiments, the target RNA-specific sequence comprises at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides having a sequence that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of another target RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the levels of the at least one target RNA, including 13214, in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. (See www.luminexcorp.com/technology/index.html). In some embodiments, total RNA is isolated from a sample and is then labeled with biotin. The labeled RNA is then hybridized to target RNA-specific capture probes (e.g., FlexmiR™ products sold by Luminex, Inc. at http://www.luminexcorp.com/products/assays/index.html) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample (total RNA or enriched small RNAs) is first polyadenylated, and is subsequently labeled with a biotinylated 3DNA™ dendrimer (i.e., a multiple-arm DNA with numerous biotin molecules bound thereto), such as those sold by Marligen Biosciences as the Vantage™ microRNA Labeling Kit, using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. See www.marligen.com/vantage-microrna-labeling-kit.html. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target RNA, including 13214, in the methods described herein is by gel electrophoresis and detection with labeled probes (e.g., probes labeled with a radioactive or chemiluminescent label), such as by Northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Varallyay, E. et al. (2008) Nature Protocols 3(2):190-196, which is incorporated herein by reference in its entirety. In some embodiments, the total RNA sample can be further purified to enrich for small RNAs. In some embodiments, target RNAs can be amplified by, e.g., rolling circle amplification using a long probe that is complementary to both ends of a target RNA (“padlocked probes”), ligation to circularize the probe followed by rolling circle replication using the target RNA hybridized to the circularized probe as a primer. See, e.g., Jonstrup, S. P. et al. (2006) RNA 12:1-6, which is incorporated herein by reference in its entirety. The amplified product can then be detected and quantified using, e.g., gel electrophoresis and Northern blotting.

In alternative embodiments, labeled probes are hybridized to isolated total RNA in solution, after which the RNA is subjected to rapid ribonuclease digestion of single-stranded RNA, e.g., unhybridized portions of the probes or unhybridized target RNAs. In these embodiments, the ribonuclease treated sample is then analyzed by SDS-PAGE and detection of the radiolabeled probes by, e.g., Northern blotting. See mirVana™ miRNA Detection Kit sold by Applied Biosystems, Inc. product literature at www.ambion.com/catalog/CatNum.php?1552.

In some embodiments, the analytical method used for detecting and quantifying the at least one target RNA, including 13214, in the methods described herein is by hybridization to a microarray. See, e.g., Liu, C. G. et al. (2004) Proc. Nat'l Acad. Sci. USA 101:9740-9744; Lim, L. P. et al. (2005) Nature 433:769-773, each of which is incorporated herein by reference in its entirety.

In some embodiments, detection and quantification of a target RNA using a microarray is accomplished by surface plasmon resonance. See, e.g., Nanotech News (2006), available at http://nano.cancer.gov/news_center/nanotech_news_{—}2006-10-30b.asp. In these embodiments, total RNA is isolated from a sample being tested. Optionally, the RNA sample is further purified to enrich the population of small RNAs. After purification, the RNA sample is bound to an addressable microarray containing probes at defined locations on the microarray. In some embodiments, the RNA is reverse transcribed to cDNA, and the cDNA is bound to an addressable microarray. In some such embodiments, the microarray comprises probes that have regions that are complementary to the cDNA sequence (i.e., the probes comprise regions that have the same sequence as the RNA to be detected). Nonlimiting exemplary 13214 capture probes comprise a region comprising a sequence selected from (for each probe, it is indicated whether the probe hybridizes to the “sense” mature RNA, or the “antisense” of the mature RNA (i.e., hybridizes to a cDNA reverse-transcribed from the RNA)):

(SEQ ID NO: 7)
5′-CTGAGTACTTTAGTTAAGGAA-3′
for sense RNA;
(SEQ ID NO: 8)
5′-TCTGAGTACTTTAGTTAAGGAA-3′
for sense RNA;
(SEQ ID NO: 9)
5′-CTGAGTACTTTAGTTAAGGAAA-3′
for sense RNA;
(SEQ ID NO: 10)
5′-TCTGAGTACTTTAGTTAAGGAAA-3′
for sense RNA;
(SEQ ID NO: 11)
5′-TTCCTTAACTAAAGTACTCAG-3′
for cDNA.
(SEQ ID NO: 12)
5′-TTCCTTAACTAAAGTACTCAGA-3′
for cDNA;
(SEQ ID NO: 13)
5′-TTTCCTTAACTAAAGTACTCAG-3′
for cDNA;
(SEQ ID NO: 14)
5′-TTTCCTTAACTAAAGTACTCAGA-3′
for cDNA.

Further nonlimiting exemplary probes comprise a region having at least 8, at least 9, at least 10, at least 11, at least 12, at least 13, at least 14, at least 15, at least 16, at least 17, or at least 18 contiguous nucleotides of a sequence selected from SEQ ID NOs: 7 to 14. A probe may further comprise at least a second region that does not comprise a sequence that is identical to at least 8 contiguous nucleotides of a sequence selected from SEQ ID NOs: 7 to 14.

Nonlimiting exemplary probes comprise a region having at least 8, at least 9, at least 10, at least 11, at least 12, at least 13, at least 14, at least 15, at least 16, at least 17, or at least 18, at least 19, at least 20, at least 25, at least 30, at least 40, at least 50, at least 60, or at least 70 contiguous nucleotides of a sequence selected from (for each probe, it is indicated whether the probe hybridizes to the “sense” RNA, or the “antisense” of the RNA (i.e., hybridizes to a cDNA reverse-transcribed from the RNA)):

(SEQ ID NO: 15)
5′-TTCTTACCTT ACTACATCAT CAATATTGTT CCTGTATACG
CCTTCAAGTC TTTCTGCAGG AAATCCCATA GCAATAATGT
TTGGATAAAT ATCTGAGTAC TTTAGTTAAG GAAAGAAAT-3′
for sense 13214 pre-miRNA;
(SEQ ID NO: 16)
5′-ATTTCTTTCC TTAACTAAAG TACTCAGATA TTTATCCAAA
CATTATTGCT ATGGGATTTC CTGCAGAAAG ACTTGAAGGC
GTATACAGGA ACAATATTGA TGATGTAGTA AGGTAAGAA-3′
for cDNA reverse-transcribed from 13214 pre-
miRNA.

In some embodiments, the probes contain one or more affinity-enhancing nucleotide analogs as discussed below, such as locked nucleic acid (“LNA”) nucleotide analogs. After hybridization to the microarray, the RNA that is hybridized to the array is first polyadenylated, and the array is then exposed to gold particles having poly-dT bound to them. The amount of bound target RNA is quantitated using surface plasmon resonance.

In some embodiments, microarrays are utilized in a RNA-primed, Array-based Klenow Enzyme (“RAKE”) assay. See Nelson, P. T. et al. (2004) Nature Methods 1(2):1-7; Nelson, P. T. et al. (2006) RNA 12(2):1-5, each of which is incorporated herein by reference in its entirety. In some embodiments, total RNA is isolated from a sample. In some embodiments, small RNAs are isolated from a sample. The RNA sample is then hybridized to DNA probes immobilized at the 5′-end on an addressable array. The DNA probes comprise, in some embodiments, from the 5′-end to the 3′-end, a first region comprising a “spacer” sequence which is the same for all probes, a second region comprising three thymidine-containing nucleosides, and a third region comprising a sequence that is complementary to a target RNA of interest, such as 13214.

After the sample is hybridized to the array, it is exposed to exonuclease I to digest any unhybridized probes. The Klenow fragment of DNA polymerase I is then applied along with biotinylated dATP, allowing the hybridized target RNAs to act as primers for the enzyme with the DNA probe as template. The slide is then washed and a streptavidin-conjugated fluorophore is applied to detect and quantitate the spots on the array containing hybridized and Klenow-extended target RNAs from the sample.

In some embodiments, the RNA sample is reverse transcribed. In some embodiments, the RNA sample is reverse transcribed using a biotin/poly-dA random octamer primer. When than primer is used, the RNA template is digested and the biotin-containing cDNA is hybridized to an addressable microarray with bound probes that permit specific detection of target RNAs. In typical embodiments, the microarray includes at least one probe comprising at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides identically present in, or complementary to a region of, a target RNA, such as 13214. After hybridization of the cDNA to the microarray, the microarray is exposed to a streptavidin-bound detectable marker, such as a fluorescent dye, and the bound cDNA is detected. See Liu C. G. et al. (2008) Methods 44:22-30, which is incorporated herein by reference in its entirety.

In some embodiments, target RNAs, including 13214, are detected and quantified in an ELISA-like assay using probes bound in the wells of microtiter plates. See Mora J. R. and Getts R. C. (2006) BioTechniques 41:420-424 and supplementary material in BioTechniques 41(4):1-5; U.S. Patent Publication No. 2006/0094025 to Getts et al., each of which is incorporated by reference herein in its entirety. In these embodiments, a sample of RNA that is enriched in small RNAs is either polyadenylated, or is reverse transcribed and the cDNA is polyadenylated. The RNA or cDNA is hybridized to probes immobilized in the wells of a microtiter plates, wherein each of the probes comprises a sequence that is identically present in, or complementary to a region of, a target RNA, such as 13214. In some embodiments, the hybridized RNAs are labeled using a capture sequence, such as a DNA dendrimer (such as those available from Genisphere, Inc., http://www.genisphere.com/about_{—}3 dna.html) that is labeled with a plurality of biotin molecules or with a plurality of horseradish peroxidase molecules, and a bridging polynucleotide that contains a poly-dT sequence at the 5′-end that binds to the poly-dA tail of the captured nucleic acid, and a sequence at the 3′-end that is complementary to a region of the capture sequence. If the capture sequence is biotinylated, the microarray is then exposed to streptavidin-bound horseradish peroxidase. Hybridization of target RNAs is detected by the addition of a horseradish peroxidase substrate such as tetramethylbenzidine (TMB) and measurement of the absorbance of the solution at 450 nM.

In still other embodiments, an addressable microarray is used to detect a target RNA using quantum dots. See Liang, R. Q. et al. (2005) Nucl. Acids Res. 33(2):e17, available at http://www.pubmedcentral.nih.gov/articlerender.fcgi?artid=548377, which is incorporated herein by reference in its entirety. In some embodiments, total RNA is isolated from a sample. In some embodiments, small RNAs are isolated from the sample. The 3′-ends of the target RNAs are biotinylated using biotin-X-hydrazide. The biotinylated target RNAs are captured on a microarray comprising immobilized probes comprising sequences that are identically present in, or complementary to a region of, target RNAs, including 13214. The hybridized target RNAs are then labeled with quantum dots via a biotin-streptavidin binding. A confocal laser causes the quantum dots to fluoresce and the signal can be quantified. In alternative embodiments, small RNAs can be detected using a colorimetric assay. In these embodiments, small RNAs are labeled with streptavidin-conjugated gold followed by silver enhancement. The gold nanoparticles bound to the hybridized target RNAs catalyze the reduction of silver ions to metallic silver, which can then be detected colorimetrically with a CCD camera

In some embodiments, detection and quantification of one or more target RNAs is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra. The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. See U.S. Patent Publication No. 2006/0292616 to Neely et al., which is hereby incorporated by reference in its entirety. In some alternative embodiments, a target RNA-specific probe can be labeled with 3 or more distinct labels selected from, e.g., fluorophores, electron spin labels, etc., and then hybridized to an RNA sample, such as total RNA, or a sample that is enriched in small RNAs.

Nonlimiting exemplary target RNA-specific probes include probes comprising sequences selected from SEQ ID NOs: 7 to 14; sequences having at least 8, at least 9, at least 10, at least 11, at least 12, at least 13, at least 14, at least 15, at least 16, at least 17, or at least 18 contiguous nucleotides of a sequence selected from SEQ ID NOs: 7 to 14; and sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16.

Optionally, the sample RNA is modified before hybridization. The target RNA/probe duplex is then passed through channels in a microfluidic device and that comprise detectors that record the unique signal of the 3 labels. In this way, individual molecules are detected by their unique signal and counted. See U.S. Pat. Nos. 7,402,422 and 7,351,538 to Fuchs et al., U.S. Genomics, Inc., each of which is incorporated herein by reference in its entirety.

In some embodiments, the detection and quantification of one or more target RNAs is accomplished by a solution-based assay, such as a modified Invader assay. See Allawi H. T. et al. (2004) RNA 10:1153-1161, which is incorporated herein by reference in its entirety. In some embodiments, the modified invader assay can be performed on unfractionated detergent lysates of cervical cells. In other embodiments, the modified invader assay can be performed on total RNA isolated from cells or on a sample enriched in small RNAs. The target RNAs in a sample are annealed to two probes which form hairpin structures. A first probe has a hairpin structure at the 5′ end and a region at the 3′-end that has a sequence that is complementary to the sequence of a region at the 5′-end of a target RNA. The 3′-end of the first probe is the “invasive polynucleotide”. A second probe has, from the 5′ end to the 3′-end a first “flap” region that is not complementary to the target RNA, a second region that has a sequence that is complementary to the 3′-end of the target RNA, and a third region that forms a hairpin structure. When the two probes are bound to a target RNA target, they create an overlapping configuration of the probes on the target RNA template, which is recognized by the Cleavase enzyme, which releases the flap of the second probe into solution. The flap region then binds to a complementary region at the 3′-end of a secondary reaction template (“SRT”). A FRET polynucleotide (having a fluorescent dye bound to the 5′-end and a quencher that quenches the dye bound closer to the 3′ end) binds to a complementary region at the 5′-end of the SRT, with the result that an overlapping configuration of the 3′-end of the flap and the 5′-end of the FRET polynucleotide is created. Cleavase recognizes the overlapping configuration and cleaves the 5′-end of the FRET polynucleotide, generates a fluorescent signal when the dye is released into solution.

4.1.5. Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot (GE Healthcare), ABI 3900 DNA Synthesizer (Applied Biosystems), and the like. Enzymatic synthesis includes, but is not limited, to producing polynucleotides by enzymatic amplification, e.g., PCR.

In some embodiments, a polynucleotide is provided that comprises at least 8, at least 9, at least 10, at least 11, at least 12, at least 13, at least 14, at least 15, at least 16, at least 17, or at least 18 contiguous nucleotides of a sequence selected from SEQ ID NOs: 7 to 14. In some embodiments, a polynucleotide is provided that comprises at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 8 and 200, between 8 and 150, between 8 and 100, between 8 and 75, between 8 and 50, between 8 and 40, or between 8 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of specifically hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). In other embodiments, the donor and acceptor are not at the ends of the FRET probe. Thus, in some embodiments, the emission spectrum of the donor moiety should overlap considerably with the absorption spectrum of the acceptor moiety.

4.1.5.1. Exemplary Polynucleotide Modifications

In some embodiments, the methods of detecting at least one target RNA described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

4.1.5.2. Exemplary Primers

In some embodiments, a primer is provided. In some embodiments, a primer is identical or complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of a target RNA, such as 13214. In some embodiments, a primer may also comprise portions or regions that are not identical or complementary to the target RNA. In some embodiments, a region of a primer that is identical or complementary to a target RNA is contiguous, such that any region of a primer that is not identical or complementary to the target RNA does not disrupt the identical or complementary region.

In some embodiments, a primer comprises a portion that is identically present in a target RNA, such as 13214. In some such embodiments, a primer that comprises a region that is identically present in the target RNA is capable of selectively hybridizing to a cDNA that has been reverse transcribed from the RNA, or to an amplicon that has been produced by amplification of the target RNA or cDNA. In some embodiments, the primer is complementary to a sufficient portion of the cDNA or amplicon such that it selectively hybridizes to the cDNA or amplicon under the conditions of the particular assay being used.

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

Nonlimiting exemplary primers include primers comprising sequences that are identically present in, or complementary to a region of, 13214, or another target RNA. Nonlimiting exemplary primers include polynucleotides comprising sequences selected from SEQ ID NOs: 7 to 14; sequences having at least 8, at least 9, at least 10, at least 11, at least 12, at least 13, at least 14, at least 15, at least 16, at least 17, or at least 18 contiguous nucleotides of a sequence selected from SEQ ID NOs: 7 to 14; and sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein. In some embodiments, a primer comprises a detectable moiety.

4.1.5.3. Exemplary Probes

In various embodiments, methods of detecting the presence of a cancer comprise hybridizing nucleic acids of a sample with a probe. In some embodiments, the probe comprises a portion that is complementary to a target RNA, such as 13214. In some embodiments, the probe comprises a portion that is identically present in the target RNA, such as 13214. In some such embodiments, a probe that is complementary to a target RNA is complementary to a sufficient portion of the target RNA such that it selectively hybridizes to the target RNA under the conditions of the particular assay being used. In some embodiments, a probe that is complementary to a target RNA is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the target RNA. In some embodiments, a probe that is complementary to a target RNA comprises a region that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the target RNA. That is, a probe that is complementary to a target RNA may also comprise portions or regions that are not complementary to the target RNA. In some embodiments, a region of a probe that is complementary to a target RNA is contiguous, such that any region of a probe that is not complementary to the target RNA does not disrupt the complementary region.

In some embodiments, the probe comprises a portion that is identically present in the target RNA, such 13214. In some such embodiments, a probe that comprises a region that is identically present in the target RNA is capable of selectively hybridizing to a cDNA that has been reverse transcribed from the RNA, or to an amplicon that has been produced by amplification of the target RNA or cDNA. In some embodiments, the probe is complementary to a sufficient portion of the cDNA or amplicon such that it selectively hybridizes to the cDNA or amplicon under the conditions of the particular assay being used. In some embodiments, a probe that is complementary to a cDNA or amplicon is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the cDNA or amplicon. In some embodiments, a probe that is complementary to a target RNA comprises a region that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the cDNA or amplicon. That is, a probe that is complementary to a cDNA or amplicon may also comprise portions or regions that are not complementary to the cDNA or amplicon. In some embodiments, a region of a probe that is complementary to a cDNA or amplicon is contiguous, such that any region of a probe that is not complementary to the cDNA or amplicon does not disrupt the complementary region.

Nonlimiting exemplary probes include probes comprising sequences set forth in SEQ ID NOS: 7 to 14, and probes comprising at least 8, at least 9, at least 10, at least 11, at least 12, at least 13, at least 14, at least 15, at least 16, at least 17, or at least 18 contiguous nucleotides of a sequence selected from SEQ ID NOs: 7 to 14. Nonlimiting exemplary probes include probes comprising sequences set forth in SEQ ID NOS: 15 and 16, and probes comprising at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16.

In some embodiments, the method of detectably quantifying one or more target RNAs comprises: (a) isolating total RNA; (b) reverse transcribing a target RNA to produce a cDNA that is complementary to the target RNA; (c) amplifying the cDNA from (b); and (d) detecting the amount of a target RNA using real time RT-PCR and a detection probe.

As described herein, in some embodiments, the real time RT-PCR detection is performed using a FRET probe, which includes, but is not limited to, a TaqMan® probe, a Molecular beacon probe and a Scorpion probe. In some embodiments, the real time RT-PCR detection and quantification is performed with a TaqMan® probe, i.e., a linear probe that typically has a fluorescent dye covalently bound at one end of the DNA and a quencher molecule covalently bound at the other end of the DNA. The FRET probe comprises a sequence that is complementary to a region of the cDNA such that, when the FRET probe is hybridized to the cDNA, the dye fluorescence is quenched, and when the probe is digested during amplification of the cDNA, the dye is released from the probe and produces a fluorescence signal. In such embodiments, the amount of target RNA in the sample is proportional to the amount of fluorescence measured during cDNA amplification.

The TaqMan® probe typically comprises a region of contiguous nucleotides having a sequence that is complementary to a region of a target RNA or its complementary cDNA that is reverse transcribed from the target RNA template (i.e., the sequence of the probe region is complementary to or identically present in the target RNA to be detected) such that the probe is specifically hybridizable to the resulting PCR amplicon. In some embodiments, the probe comprises a region of at least 6 contiguous nucleotides having a sequence that is fully complementary to or identically present in a region of a cDNA that has been reverse transcribed from a target RNA template, such as comprising a region of at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target RNA to be detected.

In some embodiments, the region of the cDNA that has a sequence that is complementary to the TaqMan® probe sequence is at or near the center of the cDNA molecule. In some embodiments, there are independently at least 2 nucleotides, such as at least 3 nucleotides, such as at least 4 nucleotides, such as at least 5 nucleotides of the cDNA at the 5′-end and at the 3′-end of the region of complementarity.

In some embodiments, Molecular Beacons can be used to detect and quantitate PCR products. Like TaqMan® probes, Molecular Beacons use FRET to detect and quantitate a PCR product via a probe having a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (See www.genelink.com/newsite/products/mbintro.asp).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g, Premier Biosoft International (See www.premierbiosoft.com/tech_notes/Scorpion.html).

In some embodiments, labels that can be used on the FRET probes include colorimetric and fluorescent labels such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides useful in the preparation of RT-PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of RT-PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target RNAs are detected in a single multiplex reaction. In some such embodiments, each probe that is targeted to a unique cDNA is spectrally distinguishable when released from the probe. Thus, each target RNA is detected by a unique fluorescence signal.

One skilled in the art can select a suitable detection method for a selected assay, e.g., a real-time RT-PCR assay. The selected detection method need not be a method described herein, and may be any method.

### 4.2. Exemplary Compositions and Kits

In another aspect, compositions are provided. In some embodiments, compositions are provided for use in the methods described herein.

In some embodiments, a composition comprises at least one polynucleotide. In some embodiments, a composition comprises at least one primer. In some embodiments, a composition comprises at least one probe. In some embodiments, a composition comprises at least one primer and at least one probe.

In some embodiments, compositions are provided that comprise at least one target RNA-specific primer. The term “target RNA-specific primer” encompasses primers that have a region of contiguous nucleotides having a sequence that is (i) identically present in a target RNA, such as 13214, or (ii) complementary to the sequence of a region of contiguous nucleotides found in a target RNA, such as 13214.

In some embodiments, compositions are provided that comprise at least one target RNA-specific probe. The term “target RNA-specific probe” encompasses probes that have a region of contiguous nucleotides having a sequence that is (i) identically present in a target RNA, such as 13214, or (ii) complementary to the sequence of a region of contiguous nucleotides found in a target RNA, such as 13214.

In some embodiments, target RNA-specific primers and probes comprise deoxyribonucleotides. In other embodiments, target RNA-specific primers and probes comprise at least one nucleotide analog. Nonlimiting exemplary nucleotide analogs include, but are not limited to, analogs described herein, including LNA analogs and peptide nucleic acid (PNA) analogs. In some embodiments, target RNA-specific primers and probes comprise at least one nucleotide analog which increases the hybridization binding energy (e.g., an affinity-enhancing nucleotide analog, discussed above). In some embodiments, a target RNA-specific primer or probe in the compositions described herein binds to one target RNA in the sample. In some embodiments, a single primer or probe binds to multiple target RNAs, such as multiple isomirs.

In some embodiments, more than one primer or probe specific for a single target RNA is present in the compositions, the primers or probes capable of binding to overlapping or spatially separated regions of the target RNA.

It will be understood, even if not explicitly stated hereinafter, that in some embodiments in which the compositions described herein are designed to hybridize to cDNAs reverse transcribed from target RNAs, the composition comprises at least one target RNA-specific primer or probe (or region thereof) having a sequence that is identically present in a target RNA (or region thereof).

In some embodiments, a composition comprises a target RNA-specific primer. In some embodiments, the target RNA-specific primer is specific for 13214. In some embodiments, a composition comprises a plurality of target RNA-specific primers for each of at least 2, at least 3, at least 4, at least 5, at least 6, at least 7, or at least 8 target RNAs.

In some embodiments, a composition comprises a target RNA-specific probe. In some embodiments, the target RNA-specific probe is specific for 13214. In some embodiments, a composition comprises a plurality of target RNA-specific probes for each of at least 2, at least 3, at least 4, at least 5, at least 6, at least 7, or at least 8 target RNAs.

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include a buffering component and/or additional components.

In some embodiments, a composition comprises one or more additional components. Additional components include, but are not limited to, salts, such as NaCl, KCl, and MgCl₂; polymerases, including thermostable polymerases; dNTPs; RNase inhibitors; bovine serum albumin (BSA) and the like; reducing agents, such as β-mercaptoethanol; EDTA and the like; etc. One skilled in the art can select suitable composition components depending on the intended use of the composition.

In some embodiments, an addressable microarray component is provided that comprises target RNA-specific probes attached to a substrate.

Microarrays for use in the methods described herein comprise a solid substrate onto which the probes are covalently or non-covalently attached. In some embodiments, probes capable of hybridizing to one or more target RNAs or cDNAs are attached to the substrate at a defined location (“addressable array”). Probes can be attached to the substrate in a wide variety of ways, as will be appreciated by those in the art. In some embodiments, the probes are synthesized first and subsequently attached to the substrate. In other embodiments, the probes are synthesized on the substrate. In some embodiments, probes are synthesized on the substrate surface using techniques such as photopolymerization and photolithography.

In some embodiments, the solid substrate is a material that is modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. In some embodiments, the substrates allow optical detection without appreciably fluorescing.

In some embodiments, the substrate is planar. In other embodiments, probes are placed on the inside surface of a tube, such as for flow-through sample analysis to minimize sample volume. In other embodiments, probes can be in the wells of multi-well plates. In still other embodiments, probes can be attached to an addressable microbead array. In yet other embodiments, the probes can be attached to a flexible substrate, such as a flexible foam, including closed cell foams made of particular plastics.

The substrate and the probe can each be derivatized with functional groups for subsequent attachment of the two. For example, in some embodiments, the substrate is derivatized with one or more chemical functional groups including, but not limited to, amino groups, carboxyl groups, oxo groups and thiol groups. In some embodiments, probes are attached directly to the substrate through one or more functional groups. In some embodiments, probes are attached to the substrate indirectly through a linker (i.e., a region of contiguous nucleotides that space the probe regions involved in hybridization and detection away from the substrate surface). In some embodiments, probes are attached to the solid support through the 5′ terminus. In other embodiments, probes are attached through the 3′ terminus. In still other embodiments, probes are attached to the substrate through an internal nucleotide. In some embodiments the probe is attached to the solid support non-covalently, e.g., via a biotin-streptavidin interaction, wherein the probe biotinylated and the substrate surface is covalently coated with streptavidin.

In some embodiments, the compositions comprise a microarray having probes attached to a substrate, wherein at least one of the probes (or a region thereof) comprises a sequence that is identically present in, or complementary to a region of, 13214. In some embodiments, in addition to a probe comprising a sequence that is identically present in, or complementary to a region of, at least one of those RNAs, a microarray further comprises at least one probe comprising a sequence that is identically present in, or complementary to a region of, another target RNA. In some embodiments, in addition to a probe comprising a sequence that is identically present in, or complementary to a region of, at least one of those RNAs, a microarray further comprises at least two, at least five, at least 10, at least 15, at least 20, at least 30, at least 50, or at least 100 probes comprising sequences that are identically present in, or complementary to regions of, other target RNAs. In some embodiments, the microarray comprises each target RNA-specific probe at only one location on the microarray. In some embodiments, the microarray comprises at least one target RNA-specific probe at multiple locations on the microarray.

As used herein, the terms “complementary” or “partially complementary” to a target RNA (or target region thereof), and the percentage of “complementarity” of the probe sequence to that of the target RNA sequence is the percentage “identity” to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and the reverse complement of the sequence of the target RNA that best aligns therewith. The percentage is calculated by counting the number of aligned bases that are identical as between the 2 sequences, dividing by the total number of contiguous nucleotides in the probe, and multiplying by 100.

In some embodiments, the microarray comprises at least one probe having a region with a sequence that is fully complementary to a target region of a target RNA. In other embodiments, the microarray comprises at least one probe having a region with a sequence that comprises one or more base mismatches when compared to the sequence of the best-aligned target region of a target RNA.

In some embodiments, the microarray comprises at least one probe having a region of at least 10, at least 11, at least 13, at least 14, at least 15, at least 16, at least 17, at least 18 contiguous nucleotides identically present in, or complementary to, 13214. In some embodiments, the microarray comprises at least one probe having a region of at least 10, at least 11, at least 13, at least 14, at least 15, at least 16, at least 17, at least 18, at least 19, at least 20, at least 21, at least 22, at least 23, at least 24, or at least 25 contiguous nucleotides with a sequence that is identically present in, or complementary to a region of, another target RNA.

In some embodiments, the microarrays comprise probes having a region with a sequence that is complementary to target RNAs that comprise a substantial portion of the human miRNome (i.e., the publicly known microRNAs that have been accessioned by others into miRBase (http://microrna.sanger.ac.uk/ at the time the microarray is fabricated), such as at least about 60%, at least about 70%, at least about 80%, at least about 90%, even at least about 95% of the human miRNome. In some embodiments, the microarrays comprise probes that have a region with a sequence that is identically present in target RNAs that comprise a substantial portion of the human miRNome, such as at least about 60%, at least about 70%, at least about 80%, at least about 90%, even at least about 95% of the human miRNome.

In some embodiments, components are provided that comprise probes attached to microbeads, such as those sold by Luminex, each of which is internally dyed with red and infrared fluorophores at different intensities to create a unique signal for each bead. In some embodiments, the compositions useful for carrying out the methods described herein include a plurality of microbeads, each with a unique spectral signature. Each uniquely labeled microbead is attached to a unique target RNA-specific probe such that the unique spectral signature from the dyes in the bead is associated with a particular probe sequence. Nonlimiting exemplary probe sequences include SEQ ID NOs: 7 to 14. Nonlimiting exemplary probe sequences include sequences having at least 8, at least 9, at least 10, at least 11, at least 12, at least 13, at least 14, at least 15, at least 16, at least 17, at least 18 contiguous nucleotides of a sequence selected from SEQ ID NOs: 7 to 14. Nonlimiting exemplary probe sequences include sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16. Nonlimiting exemplary probe sequences also include probes comprising a region that is identically present in, or complementary to, at least 8 contiguous nucleotides of 13214. Nonlimiting exemplary probe sequences also include probes comprising a region that is identically present in, or complementary to, other target RNAs.

In some embodiments, a uniquely labeled microbead has attached thereto a probe having a region with a sequence that is identically present in, or complementary to a region of at least 8 contiguous nucleotides of 13214. In some embodiments, a uniquely labeled microbead has attached thereto a probe comprising a sequence selected from SEQ ID NOs: 7 to 14. In some embodiments, a uniquely labeled microbead has attached thereto a probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NOs: 7 to 14. In some embodiments, a uniquely labeled microbead has attached thereto a probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16. In some embodiments, a uniquely labeled microbead has attached thereto a probe having a region with a sequence that is identically present in, or complementary to a region of, another target RNA.

In some embodiments, a composition is provided that comprises a plurality of uniquely labeled microbeads, wherein at least one microbead has attached thereto a probe having a region with a sequence that is identically present in, or complementary to a region of, at least 8 contiguous nucleotides of 13214. In some embodiments, a composition is provided that comprises a plurality of uniquely labeled microbeads, wherein at least one microbead has attached thereto a probe comprising a sequence selected from SEQ ID NOs: 7 to 14. In some embodiments, a composition is provided that comprises a plurality of uniquely labeled microbeads, wherein at least one microbead has attached thereto a probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NOs: 7 to 14. In some embodiments, a composition is provided that comprises a plurality of uniquely labeled microbeads, wherein at least one microbead has attached thereto a probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16. In some embodiments, a composition is provided that comprises a plurality of uniquely labeled microbeads, wherein at least one microbead has attached thereto a probe having a region with a sequence that is identically present in, or complementary to a region of, at least 8 contiguous nucleotides of 13214, and at least one microbead has attached thereto a probe having a region with a sequence that is identically present in, or complementary to a region of, another target RNA.

In some embodiments, the compositions comprise a plurality of uniquely labeled microbeads, each of which has attached thereto a unique probe having a region that is complementary to target RNAs that comprise a substantial portion of the human miRNome, such as at least about 60%, at least about 70%, at least about 80%, at least about 90%, or at least about 95% of the human miRNome. In some embodiments, the compositions comprise a plurality of uniquely labeled microbeads having attached thereto a unique probe having a region with a sequence that is identically present in target RNAs that comprise a substantial portion of the human miRNome, such as at least about 60%, at least about 70%, at least about 80%, at least about 90%, or at least about 95% of the human miRNome.

In some embodiments, compositions are provided that comprise at least one polynucleotide for detecting at least one target RNA. In some embodiments, the polynucleotide is used as a primer for a reverse transcriptase reaction. In some embodiments, the polynucleotide is used as a primer for amplification. In some embodiments, the polynucleotide is used as a primer for RT-PCR. In some embodiments, the polynucleotide is used as a probe for detecting at least one target RNA. In some embodiments, the polynucleotide is detectably labeled. In some embodiments, the polynucleotide is a FRET probe. In some embodiments, the polynucleotide is a TaqMan® probe, a Molecular Beacon, or a Scorpion probe.

In some embodiments, a composition comprises at least one FRET probe having a sequence that is identically present in, or complementary to a region of, 13214. In some embodiments, a composition comprises at least one FRET probe having a sequence selected from SEQ ID NOs: 7 to 14. In some embodiments, a composition comprises at least one FRET probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NOs: 7 to 14. In some embodiments, a composition comprises at least one FRET probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 15 and 16. In some embodiments, a composition comprises at least one FRET probe having a region with a sequence that is identically present in, or complementary to a region of, 13214, and at least one FRET probe having a region with a sequence that is identically present in, or complementary to a region of, another target RNA.

In some embodiments, a FRET probe is labeled with a donor/acceptor pair such that when the probe is digested during the PCR reaction, it produces a unique fluorescence emission that is associated with a specific target RNA. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target RNA. In some embodiments, the sequence of the FRET probe is complementary to a target region of a target RNA. In other embodiments, the FRET probe has a sequence that comprises one or more base mismatches when compared to the sequence of the best-aligned target region of a target RNA.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is identically present in, or complementary to a region of, 13214. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, 13214. In some embodiments, the FRET probe has a sequence with one, two or three base mismatches when compared to the sequence or complement of 13214.

In some embodiments, the compositions further comprise a FRET probe consisting of at least 10, at least 11, at least 13, at least 14, at least 15, at least 16, at least 17, at least 18, at least 19, at least 20, at least 21, at least 22, at least 23, at least 24, or at least 25 contiguous nucleotides, wherein the FRET probe comprises a sequence that is identically present in, or complementary to a region of, a region of another target RNA. In some embodiments, the FRET probe is identically present in, or complementary to a region of, at least at least 10, at least 11, at least 13, at least 14, at least 15, at least 16, at least 17, at least 18, at least 19, at least 20, at least 21, at least 22, at least 23, or at least 24 contiguous nucleotides of another target RNA.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target RNA-specific FRET probes and/or one or more primers for reverse transcription of target RNAs and/or one or more primers for amplification of target RNAs or cDNAs reverse transcribed therefrom.

In some embodiments, one or more of the primers and/or probes is “linear”. A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target RNA.

In some embodiments, a kit comprises one or more pairs of linear primers (a “forward primer” and a “reverse primer”) for amplification of a cDNA reverse transcribed from a target RNA, such 13214. Accordingly, in some embodiments, a first primer comprises a region of at least 4, at least 5, at least 6, at least 7, at least 8, at least 9, or at least 10 contiguous nucleotides having a sequence that is identical to the sequence of a region of at least 4, at least 5, at least 6, at least 7, at least 8, at least 9, or at least 10 contiguous nucleotides at the 5′-end of a target RNA. Furthermore, in some embodiments, a second primer comprises a region of at least 4, at least 5, at least 6, at least 7, at least 8, at least 9, or at least 10 contiguous nucleotides having a sequence that is complementary to the sequence of a region of at least 4, at least 5, at least 6, at least 7, at least 8, at least 9, or at least 10 contiguous nucleotides at the 3′-end of a target RNA. In some embodiments, the kit comprises at least a first set of primers for amplification of a cDNA that is reverse transcribed from 13214. In some embodiments, the kit further comprises at least a second set of primers for amplification of a cDNA that is reverse transcribed from another target RNA.

In some embodiments, the kit comprises at least two, at least five, at least 10, at least 15, at least 20, at least 25, at least 30, at least 40, at least 50, at least 60, at least 75, or at least 100 sets of primers, each of which is for amplification of a cDNA that is reverse transcribed from a different target RNA, including 13214. In some embodiments, the kit comprises at least one set of primers that is capable of amplifying more than one cDNA reverse transcribed from a target RNA in a sample.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described herein. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the compositions described herein also comprise probes, and in the case of RT-PCR, primers, that are specific to one or more housekeeping genes for use in normalizing the quantities of target RNAs. Such probes (and primers) include those that are specific for one or more products of housekeeping genes selected from U6 snRNA, ACTB, B2M, GAPDH, GUSB, HPRT1, PPIA, RPLP, RRN18S, TBP, TUBB, UBC, YWHA (TATAA), PGK1, and RPL4.

In some embodiments, the kits for use in real time RT-PCR methods described herein further comprise reagents for use in the reverse transcription and amplification reactions. In some embodiments, the kits comprise enzymes such as reverse transcriptase, and a heat stable DNA polymerase, such as Taq polymerase. In some embodiments, the kits further comprise deoxyribonucleotide triphosphates (dNTP) for use in reverse transcription and amplification. In further embodiments, the kits comprise buffers optimized for specific hybridization of the probes and primers.

4.2.1. Exemplary Normalization of RNA Levels

In some embodiments, quantitation of target RNA levels requires assumptions to be made about the total RNA per cell and the extent of sample loss during sample preparation. In order to correct for differences between different samples or between samples that are prepared under different conditions, the quantities of target RNAs in some embodiments are normalized to the levels of at least one endogenous housekeeping gene.

Appropriate genes for use as reference genes in the methods described herein include those as to which the quantity of the product does not vary between normal and cancerous cells, or between different cell lines or under different growth and sample preparation conditions. In some embodiments, endogenous housekeeping genes useful as normalization controls in the methods described herein include, but are not limited to, U6 snRNA, RNU44, RNU 48, and U47. In typical embodiments, the at least one endogenous housekeeping gene for use in normalizing the measured quantity of RNAs is selected from U6 snRNA, U6 snRNA, RNU44, RNU 48, and U47. In some embodiments, one housekeeping gene is used for normalization. In some embodiments, more than one housekeeping gene is used for normalization.

In some embodiments, a spike-in control polynucleotide is added to a patient sample, such as a serum sample, as a control. A nonlimiting exemplary spike-in control is CelmiR-39. In some embodiments, a spike-in control is used to correct for variations in RNA purification from the sample, such as serum. In some embodiments, the spike-in control is detected in the same, or a similar, assay as the target RNA(s). One skilled in the art can select a suitable spike-in control depending on the application.

4.2.2. Exemplary Qualitative Methods

In some embodiments, methods comprise detecting a qualitative change in a target RNA profile generated from a clinical sample as compared to a normal target RNA profile (in some exemplary embodiments, a target RNA profile of a control sample). Some qualitative changes in the RNA profile are indicative of the presence of cancer in the subject from which the clinical sample was taken. Various qualitative changes in the RNA profile are indicative of the propensity to proceed to cancer. The term “target RNA profile” refers to a set of data regarding the concurrent levels of a plurality of target RNAs in the same sample.

In some embodiments, at least one of the target RNAs of the plurality of target RNAs is 13214. In some embodiments, the plurality of target RNAs comprises at least one, at least two, at least five, at least 10, at least 15, at least 20, at least 25, at least 30, at least 40, at least 50, at least 60, at least 75, or at least 100 additional target RNAs. In some embodiments, a target RNA, in its mature form, comprises fewer than 30 nucleotides. In some embodiments, a target RNA is a microRNA. In some embodiments, a target RNA is a small cellular RNA.

Qualitative data for use in preparing target RNA profiles is obtained using any suitable analytical method, including the analytical methods presented herein.

In some embodiments, for example, concurrent RNA profile data are obtained using, e.g., a microarray, as described herein. Thus, in addition to use for quantitatively determining the levels of specific target RNAs as described herein, a microarray comprising probes having sequences that are complementary to a substantial portion of the miRNome may be employed to carry out target RNA profiling, for analysis of target RNA expression patterns.

According to the RNA profiling method, in some embodiments, total RNA from a sample from a subject suspected of having cancer is quantitatively reverse transcribed to provide a set of labeled polynucleotides complementary to the RNA in the sample. The polynucleotides are then hybridized to a microarray comprising target RNA-specific probes to provide a hybridization profile for the sample. The result is a hybridization profile for the sample representing the target RNA profile of the sample. The hybridization profile comprises the signal from the binding of the polynucleotides reverse transcribed from the sample to the target RNA-specific probes in the microarray. In some embodiments, the profile is recorded as the presence or absence of binding (signal vs. zero signal). In some embodiments, the profile recorded includes the intensity of the signal from each hybridization. The profile is compared to the hybridization profile generated from a normal, i.e., noncancerous, or in some embodiments, a control sample. An alteration in the signal is indicative of the presence of cancer in the subject.

### 4.3. Exemplary Additional Target RNAs

In some embodiments, in combination with detecting 13214, a method comprises detecting one or more additional target RNAs. Additional target RNAs include, but are not limited to, microRNAs, other small cellular RNAs, and mRNAs. In some embodiments, one or more additional target RNAs that have been shown to correlate with cancer in general, or a particular type or stage of cancer, are selected.

In some embodiments, the methods described herein further comprise detecting chromosomal codependents, i.e., target RNAs clustered near each other in the human genome which tend to be regulated together. Accordingly, in further embodiments, the methods comprise detecting the expression of one or more target RNAs, each situated within the chromosome no more than 50,000 bp from the chromosomal location of 13214.

### 4.4. Pharmaceutical Compositions and Methods of Treatment

In some embodiments, the disclosure relates to methods of treating cancer in which expression of a target RNA is deregulated, e.g., either down-regulated or up-regulated in the cancer cells of an individual. In some embodiments, the disclosure relates to methods of treating cancer in which levels of a target RNA are altered relative to normal cells or serum, e.g., either lower or higher in the cancer cells of an individual. When at least one isolated target RNA is up-regulated in the cancer cells, the method comprises administering to the individual an effective amount of at least one compound that inhibits the expression of the at least one target RNA, such that proliferation of cancer cells is inhibited. Alternatively, in some embodiments, when at least one target RNA is up-regulated in the cancer cells, the method comprises administering to the individual an effective amount of at least one compound that inhibits the activity of the at least one target RNA, such that proliferation of cancer cells is inhibited. Such a compound may be, in some embodiments, a polynucleotide, including a polynucleotide comprising modified nucleotides.

When at least one target RNA is down-regulated in the cancer cells, such as 13214, the method comprises administering an effective amount of an isolated target RNA (i.e., in some embodiments, a target RNA that is chemically synthesized, recombinantly expressed or purified from its natural environment), or an isolated variant or biologically-active fragment thereof, such that proliferation of cancer cells in the individual is inhibited.

The disclosure further provides pharmaceutical compositions for treating cancer. In some embodiments, the pharmaceutical compositions comprise at least one isolated target RNA, or an isolated variant or biologically-active fragment thereof, and a pharmaceutically-acceptable carrier. In some embodiments, the at least one isolated target RNA corresponds to a target RNA, such as 13214, that is present at decreased levels in cancer cells relative to normal levels (in some exemplary embodiments, relative to the level of the target RNA in a control sample).

In some embodiments the isolated target RNA is identical to an endogenous wild-type target RNA gene product that is down-regulated in the cancer cell. In some embodiments, the isolated target RNA is a variant target RNA or biologically active fragment thereof. As used herein, a “variant” refers to a target RNA gene product that has less than 100% sequence identity to the corresponding wild-type target RNA, but still possesses one or more biological activities of the wild-type target RNA (e.g., ability to inhibit expression of a target RNA molecule and cellular processes associated with cancer). A “biologically active fragment” of a target RNA is a fragment of the target RNA gene product that possesses one or more biological activities of the wild-type target RNA. In some embodiments, the isolated target RNA can be administered with one or more additional anti-cancer treatments including, but not limited to, chemotherapy, radiation therapy and combinations thereof. In some embodiments, the isolated target RNA is administered concurrently with additional anti-cancer treatments. In some embodiments, the isolated target RNA is administered sequentially to additional anti-cancer treatments.

In some embodiments, the pharmaceutical compositions comprise at least one compound that inhibits the expression or activity of a target RNA. In some embodiments, the compound is specific for one or more target RNAs, the levels of which are increased in cancer cells relative to normal levels (in some exemplary embodiments, relative to the level of the target RNA in a control sample).

In some embodiments, the target RNA inhibitor is selected from double-stranded RNA, antisense nucleic acids and enzymatic RNA molecules. In some embodiments, the target RNA inhibitor is a small molecule inhibitor. In some embodiments, the target RNA inhibitor can be administered in combination with other anti-cancer treatments, including but not limited to, chemotherapy, radiation therapy and combinations thereof. In some embodiments, the target RNA inhibitor is administered concurrently with other anti-cancer treatments. In some embodiments, the target RNA inhibitor is administered sequentially to other anti-cancer treatments.

In some embodiments, a pharmaceutical composition is formulated and administered according to Semple et al., Nature Biotechnology advance online publication, 17 Jan. 2010 (doi:10.1038/nbt.1602)), which is incorporated by reference herein in its entirety for any purpose.

The terms “treat,” “treating” and “treatment” as used herein refer to ameliorating symptoms associated with cancer, including preventing or delaying the onset of symptoms and/or lessening the severity or frequency of symptoms of the cancer.

The term “effective amount” of a target RNA or an inhibitor of target RNA expression or activity is an amount sufficient to inhibit proliferation of cancer cells in an individual suffering from cancer. An effective amount of a compound for use in the pharmaceutical compositions disclosed herein is readily determined by a person skilled in the art, e.g., by taking into account factors such as the size and weight of the individual to be treated, the stage of the disease, the age, health and gender of the individual, the route of administration and whether administration is localized or systemic.

In addition to an isolated target RNA or a target RNA inhibitor, or a pharmaceutically acceptable salt thereof, the pharmaceutical compositions disclosed herein further comprise a pharmaceutically acceptable carrier, including but not limited to, water, buffered water, normal saline, 0.4% saline, 0.3% glycine, and hyaluronic acid. In some embodiments, the pharmaceutical compositions comprise an isolated target RNA or a target RNA inhibitor that is encapsulated, e.g., in liposomes. In some embodiments, the pharmaceutical compositions comprise an isolated target RNA or a target RNA inhibitor that is resistant to nucleases, e.g., by modification of the nucleic acid backbone as described above in Section 4.1.5. In some embodiments, the pharmaceutical compositions further comprise pharmaceutically acceptable excipients such as stabilizers, antioxidants, osmolality adjusting agents and buffers. In some embodiments, the pharmaceutical compositions further comprise at least one chemotherapeutic agent, including but not limited to, alkylating agents, anti-metabolites, epipodophyllotoxins, anthracyclines, vinca alkaloids, plant alkaloids and terpenoids, monoclonal antibodies, taxanes, topoisomerase inhibitors, platinum compounds, protein kinase inhibitors, and antisense nucleic acids.

Pharmaceutical compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Methods of administration include, but are not limited to, oral, parenteral, intravenous, oral, and by inhalation.

The following examples are for illustration purposes only, and are not meant to be limiting in any way.

### 5. EXAMPLES

### 5.1 Example 1

### Detection of Cancer with 13214

Acute lymphocytic leukemia (ALL, or acute lymphoblastic leukemia) is a type of cancer of the blood and bone marrow. ALL progresses rapidly, creating immature blood cells rather than mature ones. The “lymphocytic” in acute lymphocytic leukemia refers to the white blood cells called lymphocytes, which ALL affects. ALL is the most common cancer diagnosed in children and represents 23% of cancer diagnoses among children younger than 15 years. ALL occurs at an annual rate of approximately 30 to 40 cases per million people in the United States. Approximately 2,900 children and adolescents younger than 20 years are diagnosed with ALL each year in the United States. A sharp peak in ALL incidence is observed among children aged 2 to 3 years (>80 cases per million per year), with rates decreasing to 20 cases per million for ages 8 to 10 years. The incidence of ALL among children aged 2 to 3 years is approximately fourfold greater than that for infants and is nearly tenfold greater than that for adolescents aged 16 to 21 years. Over the past 25 years, there has been a gradual increase in the incidence of ALL.

Dramatic improvements in survival have been achieved in children and adolescents with cancer. Between 1975 and 2002, childhood cancer mortality has decreased by more than 50%. For ALL, the 5-year survival rate has increased over the same time from 60% to 89% for children younger than 15 years and from 28% to 50% for adolescents aged 15 to 19 years. Childhood and adolescent cancer survivors require close follow-up because cancer therapy side effects may persist or develop months or years after treatment. Acute lymphoblastic leukemia can also occur in adults, though the chance of a cure is greatly reduced.

### Selected Cohort

Patient samples were collected at Vilnius University Children Hospital, Oncohematology Department (Vilnius, Lithuania), from January 2010 to May 2011. The study population consisted of pediatric oncology patients presenting to the hospital with the diagnosis of neutropenia and fever. Neutropenia was defined as an absolute neutrophil count (ANC) less than 0.5×10⁹/L at the onset of a fever. Fever was defined as an axillary body temperature of more than 38.5° C. in one measurement or of more than 38° C. in two repeat measurements during a six-hour period. None of the included patients were administered antibiotics before enrolment.

All patients underwent treatment with cytotoxic chemotherapy (exclusion criteria included fever more than 24 hours before admission to the hospital and antibiotic therapy in the past 72 hours). There were 30 females and 27 males with a median age of 7 years (range 1-18 years). Informed consent, after verbal and written information provision, was obtained from all patients. Permission for this study was provided by the Regional Committee of Bioethics.

Serum samples were collected during 36 fever episodes in a total of 53 oncology patients. The cancers represented included acute lymphoblastic leukemia (n=41), acute myeloblastic leukemia (n=5), non-Hodgkin's lymphoma (n=3), and non-hematologic malignancies (n=5). All patients underwent treatment with cytotoxic chemotherapy (exclusion criteria included fever more than 24 hours before admission to the hospital and antibiotic therapy in the past 72 hours). There were 30 females and 27 males with a median age of 7 years (range 1-18 years). Informed consent, after verbal and written information provision, was obtained from all patients. Permission for this study was provided by the Regional Committee of Bioethics.

Samples from 10 healthy donors were included in the study and acquired from Asterand (Royston, Herts, UK). Samples from an additional 20 healthy donors were obtained from Clinique de l'Union (Toulouse, France).

### Sampling

Venous blood samples were collected into 5 mL Vacutest polypropylene tubes with K3 EDTA (Kima Company, Arzergrande, Italy). The tubes were centrifuged at 2000×g for 10 minutes to separate the plasma, and the separated plasma was stored in Eppendorf tubes at −20° C. until evaluated. The first blood sample was taken on admission (day 1), and febrile neutropenia was confirmed to the patient before commencing antimicrobial treatment. The remaining sample was taken after 18 to 24 hours (day 2).

### RNA Extraction

RNA extraction was performed using miRNAeasy columns (Qiagen, USA) as described by the manufacturer. A spike-in control (cel-miR-39) was used for quality assessment of the extraction. Total RNA was eluted in a final volume of 50 μl of RNAse-free water.

qRT-PCR Analysis

MiRNA levels were detected by qRT-PCR using the ABi Taqman custom designs primers (Life Technologies, Foster City, Calif.) according to the manufacturer's instructions. Raw Ct values were used for analysis. Where indicated, the fold change of the miRNA was calculated from the equation 2^{-ΔCT}, where ΔCT=Mean Ct_{miRNA-A}−Mean Ct_{miRNA-B} (where Ct is the threshold cycle for a sample). The relative abundance of 13214 was calculated as the ratio of the value from cancer group to the value from controls (healthy) producing a fold change value.

### Statistical Analysis

Data was analysed using JMP 10.0 (SAS company). Briefly the non-parametric test Kruskas-Wallis followed by the Chi-square approximation was run to measure the variance between the 5 groups. When a p-value<0.05 is observed a multiple comparison (Wilcoxon test) is applied to identify the groups that are different. When a pvalue<0.05 the group pair is considered to be significantly different between the group pairs.

### Results

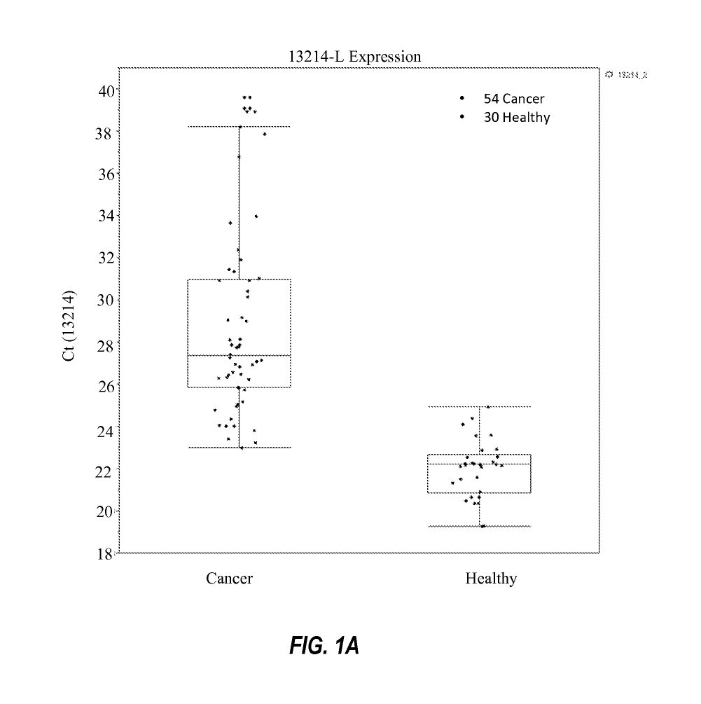

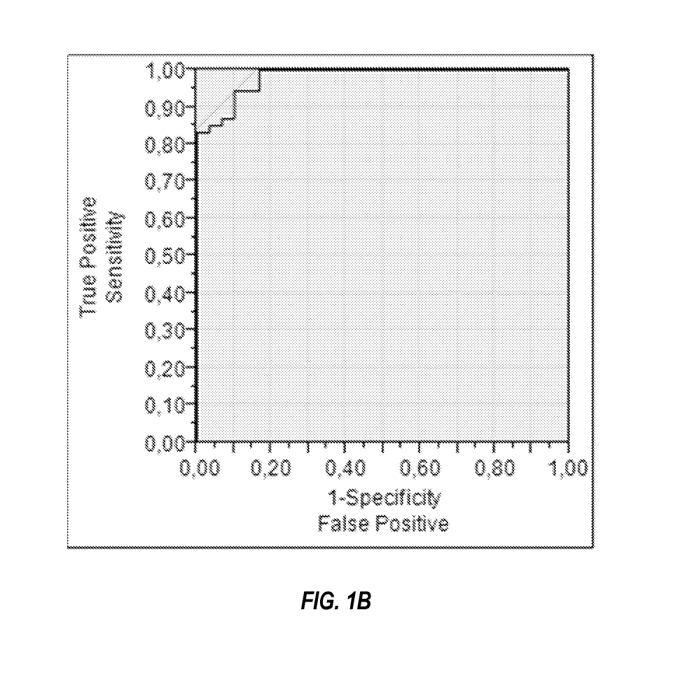

FIG. 1A shows the Ct values for all patients in the cancer groups (n=54) and all patients in the healthy group (n=30). On average, patients in the cancer group had lower levels of 13214 than the individuals in the healthy group. Applying a statistical analysis, the cancer group had statistically significant lower levels of 13214 than the healthy group (p<0.001). FIG. 1B shows a receiver operating characteristic (ROC) plot of sensitivity versus specificity for the data in FIG. 1A. The area-under-the-curve (AUC) was 0.98.

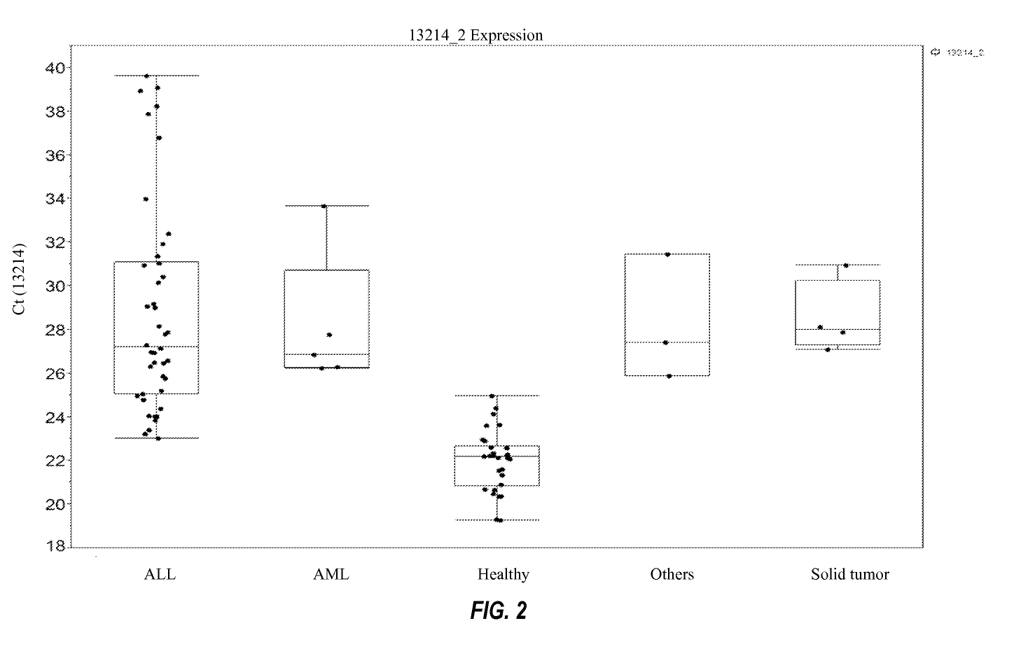

The cancer patients were then separated into their respective cancer groups for analysis. FIG. 2 shows the Ct values for patients in each cancer group, as well as the Ct values for the healthy group. Relative to the healthy group, 13214 levels were lower in acute lymphoblastic leukemia (ALL, n=41), acute myeloblastic leukemia (n=5), non-Hodgkin's lymphoma (Others, n=3), and non-hematologic malignancies (Solid tumor, n=5). Table 1 shows the statistical significance between 13214 levels for each pair of conditions in FIG. 2.

TABLE 1
Statistical significance between 13214 levels

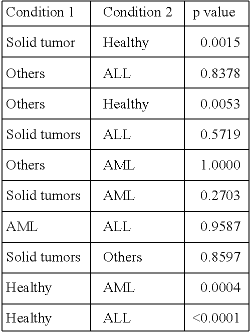

Differences in 13214 levels between healthy individuals and each group (ALL, AML, solid tumor, and others) were statistically significant (p<0.05, shaded in table).

The fold-change between the healthy group and each cancer group was also determined, and is shown in Table 2.

TABLE 2
13214 fold-change between healthy individuals and cancer patients

| | | | FC |
|---|---|---|---|
| Group | Median (Cts) | delta Ct (to healthy) | (compared to healthy) |
| Cancer | 27.35 | 5.15 | 35.51 |
| ALL | 27.21 | 5.01 | 32.22 |
| AML | 26.86 | 4.66 | 25.28 |
| Others | 27.42 | 5.22 | 37.27 |
| Solid Tumor | 28.01 | 5.81 | 55.91 |
| Healthy | 22.20 | N/A | N/A |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

What is claimed is:

1. A method for detecting the presence of cancer in a subject, the method comprising detecting the level of 13214 in a sample from the subject, wherein detection of a level of 13214 that is lower than a normal level of 13214, indicates the presence of cancer in the subject.

2. A method for detecting the presence of cancer in a subject, the method comprising detecting the level of 13214 in a sample from the subject, and comparing the level of the 13214 in the sample to a normal level of 13214, wherein detection of a level of 13214 that is lower than a normal level of 13214 indicates the presence of cancer in the subject.

3. A method of facilitating the diagnosis of cancer in a subject or monitoring therapy in a cancer patient, comprising detecting the level of 13214 in a sample from the subject, and communicating the results of the detection to a medical practitioner for the purpose of determining whether the subject has cancer or monitoring therapy in the cancer patient.

4. A method of monitoring response to therapy in a cancer patient, comprising detecting the level of 13214, in a first sample from the subject taken at a first time point, and comparing the level of 13214 to the level of 13214 in a second sample from the patient taken at a second time point, wherein the second time point is prior to the first time point, and wherein an increase in the level of 13214 in the first sample relative to the second sample, indicates that the cancer patient is responding to therapy.

5. A method for detecting the presence of cancer in a subject, comprising obtaining a sample from the subject, providing the sample to a laboratory for detection of the level of 13214 in the sample, receiving from the laboratory a communication indicating the level of 13214, wherein detection of a level of 13214 that is lower than a normal level of 13214, indicates the presence of cancer in the subject.

6. A method for monitoring response to therapy in a cancer patient, comprising obtaining a first sample from the subject at a first time point, providing the first sample to a laboratory for detection of the level of 13214, in the sample, receiving from the laboratory a communication indicating the level of 13214, comparing the level of 13214 in the first sample to the level of 13214 in a second sample that was taken at a second time point, wherein the second time point is prior to the first time point, wherein an increase in the level of 13214 in the first sample relative to the second sample, indicates that the cancer patient is responding to therapy.

7. The method of any one of claims 1 to 8, wherein the detecting comprises hybridizing at least one polynucleotide comprising at least 8 contiguous nucleotides, at least 10 contiguous nucleotides, at least 12 contiguous nucleotides, at least 14 contiguous nucleotides, or at least 16 contiguous nucleotides of a sequence selected from SEQ ID NOs: 7 to 14 to RNA from the sample or cDNA reverse-transcribed from RNA from the sample, and detecting a complex comprising a polynucleotide and a 13214 RNA or cDNA reverse transcribed therefrom.

8. The method of any one of the preceding claims, wherein 13214 is selected from mature 13214, a mature 13214 isomir, pre-13214, and combinations thereof.

9. The method of any one of the preceding claims, wherein 13214 is 13214-L.

10. The method of any one of the preceding claims, wherein 13214 has a sequence selected from SEQ ID NOs: 1 to 4.

11. The method of any one of the preceding claims, wherein the sample is selected from a tissue sample and a bodily fluid.

12. The method of claim 11, wherein the bodily fluid is selected from blood, urine, sputum, saliva, mucus, and semen.

13. The method of claim 12, wherein the sample is a blood sample.

14. The method of claim 13, wherein the blood sample is a serum sample.

15. The method of claim 13, wherein the blood sample is a plasma sample.

16. The method of any one of the preceding claims, wherein the cancer is selected from breast cancer, endometrial cancer, uterine cancer, ovarian cancer, cervical cancer, prostate cancer, leukemia, lymphoma, glioma, glioblastoma, melanoma, lung cancer, non-small cell lung cancer, liver cancer, bladder cancer, kidney cancer, pancreatic cancer, stomach cancer, adrenal pheochromocytoma, colon cancer, intestinal cancer, thyroid cancer, and skin cancer.

17. The method of any one of the preceding claims, wherein the cancer is a leukemia.

18. The method of claim 18, wherein the cancer is selected from acute lymphoblastic leukemia and acute myeloblastic leukemia.

19. The method of any one of the preceding claims, wherein the detecting comprises quantitative RT-PCR.

20. Use of 13214, for detecting the presence of cancer in a subject, or for monitoring therapy in a cancer patient.

21. An oligonucleotide that comprises at least eight contiguous nucleotides that are complementary to 13214, wherein the oligonucleotide is between 8 and 200, between 8 and 150, between 8 and 100, between 8 and 75, between 8 and 50, between 8 and 40, or between 8 and 30 nucleotides long, for detecting cancer in a subject.

22. An oligonucleotide that comprises at least eight contiguous nucleotides that are complementary to a cDNA reverse-transcribed from 13214, wherein the oligonucleotide is between 8 and 200, between 8 and 150, between 8 and 100, between 8 and 75, between 8 and 50, between 8 and 40, or between 8 and 30 nucleotides long, for detecting cancer in a subject.
